(19) United States
(12) Patent Application Publication
(10) Pub. No.: US 2024/0132604 A1
(43) Pub. Date: Apr. 25, 2024

(54) CHEMOKINE RECEPTOR 8 (CCR8) ANTIBODIES

(71) Applicant: iBio, Inc., Bryan, TX (US)

(72) Inventors: Dillon Phan, La Jolla, CA (US); Tom Sih-Yuan Hsu, Chino, CA (US); Tam Thi Thanh Phuong, San Diego, CA (US); Matthew P. Greving, Rancho Santa Fe, CA (US); Alexander Tomoaki Taguchi, San Diego, CA (US); Cory Schwartz, San Diego, CA (US); Jiang Chen, San Diego, CA (US); Gao Liu, San Diego, CA (US); Martin Brenner, San Diego, CA (US); Matthew William Dent, San Diego, CA (US); Cody Allen Moore, Del Mar, CA (US)

(21) Appl. No.: 18/489,983

(22) Filed: Oct. 18, 2023

Related U.S. Application Data
(60) Provisional application No. 63/417,507, filed on Oct. 19, 2022.
(60) Provisional application No. 63/519,004, filed on Aug. 11, 2023.

(51) Int. Cl. C07K 16/28; A61P 35/00

(52) U.S. Cl. CPC C07K 16/2866; A61P 35/00; A61K 2039/505

(57) ABSTRACT

Provided herein are anti-CCR8 antibodies or antigen binding fragment thereof, which bind to CCR8, wherein the CCR8 is a human CCR8 and the antibody does not bind human CCR4. The anti-CCR8 antibodies or antigen binding fragment of the disclosure are useful for the treatment of cancer diseases through the elimination of regulatory T cells. Also provided herein are methods of use for the anti-CCR8 antibodies or antigen binding fragment thereof.

### CROSS-REFERENCE TO RELATED APPLICATIONS

This application claims priority to U.S. Provisional Application Ser. No. 63/417,507, filed Oct. 19, 2022 and U.S. Provisional Application Ser. No. 63/519,004, filed Aug. 11, 2023 the entire contents of which are incorporated herein by reference.

### TECHNICAL FIELD OF THE INVENTION

This document relates to materials and methods for treating cancer, and particularly to the use of anti-CCR8 antibodies to reduce or eliminate cells that express CCR8, and to treat cancer.

### INCORPORATION-BY-REFERENCE OF MATERIALS FILED ON COMPACT DISC

The present application includes a Sequence Listing which has been submitted in XML format via EFS-Web and is hereby incorporated by reference in its entirety. Said XML copy, created on Oct. 19, 2023, is named IBIO:1037.xml and is 117,510 bytes in size.

### BACKGROUND OF THE INVENTION

Without limiting the scope of the invention, its background is described in connection with the elimination of cells that express CCR8.

On such is U.S. Pat. No. 11,427,640, issued to Berndt, et al., entitled, “CCR8 Antibodies for Therapeutic Applications”. These inventors are said to teach the generation of antibodies that specifically bind chemokine receptors, such as CC or CXC chemokine receptors. The isolated sulfated polypeptides and conjugates thereof can be used as antigens or for off target panning to facilitate the generation of anti-human, anti-cynomolgus, and/or anti-mouse chemokine receptor antibodies, e.g., for the generation of antibodies with fully human CDRs and/or other favorable properties for therapeutic use. These inventors also teach that the antibodies specifically binding to human, cynomolgus and/or murine CCR8 with properties for therapeutic use, such as cross-reactive antibodies, fully human antibodies, low internalizing (including non-internalizing) antibodies, and antibodies that efficiently inducing ADCC and/or ADCP in Treg cells.

Another such invention is taught in U.S. Patent Application Publication No. US 20220195057 A1, filed by Li and Huang, and entitled “Anti-CCR8 Monoclonal Antibodies and Uses Thereof”. This application is said to provide antibodies or fragment thereof having binding specificity to the human chemokine (C—C motif) receptor 8 (CCR8) protein. These antibodies are capable of binding to CCR8 at high affinity and can mediate antibody-dependent cellular cytotoxicity (ADCC).

Despite these advances, what are needed are novel antibodies, methods, and uses that specifically bind to CCR8 without binding closely related members of the chemokine receptor families, such as CC or CXC chemokine receptors.

### SUMMARY OF THE INVENTION

As embodied and broadly described herein, an aspect of the present disclosure relates to an anti-CCR8 antibody or antigen binding fragment thereof, wherein the antibody or antigen binding fragment comprises: a heavy chain variable domain (VH) complementarity determining region (CDR) 1 comprising an amino acid sequence of any one of the following SEQ ID NOS: 1, 49, 67, 73, 79, or 85; and a VH CDR2 comprising the amino acid sequence of any one of the following SEQ ID Nos: 2, 50, 68, 74, 80, or 86; and a VH CDR3 comprising the amino acid sequence of any one of the following SEQ ID NOS: 3, 51, 69, 75, 81, or 87; and a light chain variable domain (VL) CDR1 comprising the amino acid sequence of any one of the following SEQ ID Nos: 4, 52, 55, 58, 61, 64, 70, 76, 82, or 88; and a VL CDR2 comprising the amino acid sequence of any one of the following SEQ ID NOS: 5, 53, 56, 59, 62, 65, 71, 77, 83, or 89; and a VL CDR3 comprising the amino acid sequence of any one of the following SEQ ID Nos: 6, 54, 57, 60, 63, 66, 72, 78, 84, or 90. In one aspect, the antibody comprises: a VH comprising the amino acid sequence of any one of the following SEQ ID NOS: 7, 8, 9, 10, 11, 12, 13, 14, or 15, respectively; and a VL comprising the amino acid sequence of any one of the following SEQ ID NOs: 16, 17, 18, 19, 20, 21, 22, 23, or 24, respectively. In another aspect, the antibody comprises: a VH encoded by a nucleic acid sequence having at least 95, 96, 97, 98, 99, or 100% sequences identity to any one of the following SEQ ID NOS: 25, 26, 27, 28, 29, 30, 31, 32, or 33; and a VL encoded by a nucleic acid sequence having at least 95, 96, 97, 98, 99, or 100% sequences identity to any one of the following SEQ ID NOS: 34, 35, 36, 37, 18, 29, 40, 41, or 42. In another aspect, the antibody is a monoclonal, bispecific, multivalent, multi-specific, diabody, chimeric, scFv antibody, or fragments thereof. In another aspect, the antibody is fused to an Fc domain of any one of the following: human IgG1, human IgG2, human IgG3, and human IgG4. In another aspect, the antibody is a full-length antibody that is afucosylated. In another aspect, the nucleic acid sequence is optimized for expression in a bacterial, fungal, mammalian, insect, or plant cell. In another aspect, the antibody or antigen binding fragment does not bind CCR4. In another aspect, the heavy chain CDRs are SEQ ID NOS: 1, 2 and 3, and the light chain CDRs are SEQ ID NOS: 4, 5, and 6.

As embodied and broadly described herein, an aspect of the present disclosure relates to a method of treating a disease in a subject in need thereof, comprising administering to the subject a therapeutically effective amount of the antibody described hereinabove. In another aspect, the disease is cancer. In another aspect, the cancer is infiltrated with regulatory T cells. In another aspect, the subject is human.

As embodied and broadly described herein, an aspect of the present disclosure relates to a method of making an anti-CCR8 antibody or antigen binding fragment comprising expressing in the antibody or antigen binding fragment in a cell or in vitro, wherein the antibody or antigen binding fragment comprises: a heavy chain variable domain (VH) complementarity determining region (CDR) 1 comprising the amino acid sequence of any one of the following SEQ ID NOS: 1, 49, 67, 73, 79, or 85; and a VH CDR2 comprising the amino acid sequence of any one of the following SEQ ID NOS: 2, 50, 68, 74, 80, or 86; and a VH CDR3 comprising the amino acid sequence of any one of the following SEQ ID Nos: 3, 51, 69, 75, 81, or 87; and a light chain variable domain (VL) CDR1 comprising an amino acid sequence of any one of the following SEQ ID NOS: 4, 52, 55, 58, 61, 64, 70, 76, 82, or 88; and a VL CDR2 comprising the amino acid sequence of any one of the following SEQ ID NOS: 5, 53, 56, 59, 62, 65, 71, 77, 83, or 89; and a VL CDR3 comprising the amino acid sequence of any one of the following SEQ ID NOS: 6, 54, 57, 60, 63, 66, 72, 78, 84, or 90. In one aspect, the cell is a bacterial, fungal, human, plant, or insect cell. In another aspect, the antibody is a monoclonal, bispecific, multivalent, multi-specific, diabody, chimeric, scFv antibody, or fragments thereof. In another aspect, the antibody or antigen-binding fragment is afucosylated. In another aspect, the antibody or antigen-binding fragment does not bind CCR4.

As embodied and broadly described herein, an aspect of the present disclosure relates to a nucleic acid comprising an anti-CCR8 antibody or antigen-binding fragment comprising: a heavy chain variable domain encoding polynucleotide having at least 95, 96, 97, 98, 99, or 100% sequences identify to SEQ ID NOS: 25, 26, 27, 28, 29, 30, 31, 32, or 33; and a light chain variable domain encoding polynucleotide having at least 95, 96, 97, 98, 99, or 100% sequences identify to SEQ ID NOS: 34, 35, 36, 37, 38, 39, 40 41, or 42. In one aspect, the antibody is a monoclonal, bispecific, multivalent, multi-specific, diabody, chimeric, scFv antibody, or fragments thereof. In another aspect, the antibody binding domain is fused to an Fc domain of any one of the following: human IgG1, human IgG2, human IgG3, and human IgG4. In another aspect, the nucleic acid sequence is optimized for expression in a bacterial, fungal, mammalian, insect, or plant cell. In another aspect, the antibody or antigen binding fragment does not bind CCR4.

As embodied and broadly described herein, an aspect of the present disclosure relates to a vector comprising the nucleic acid disclosed hereinabove. As embodied and broadly described herein, an aspect of the present disclosure relates to a host cell comprising nucleic acid the vector disclosed hereinabove.

### BRIEF DESCRIPTION OF THE DRAWINGS

For a more complete understanding of the features and advantages of the present invention, reference is now made to the detailed description of the invention along with the accompanying figures and in which:

FIGS. 1A and 1B are flow cytometry graphs that show cell binding of hybridoma supernatant for immunized hit clone 1E6, which was identified from CCR8 MEM-ELISA binding and cell binding hybridoma screen. FIG. 1A shows clone 1E6 binds CCR8 expressing cells. FIG. 1B shows clone 1E6 binds CCR8 specifically vs. the most closely related CCR4 GPCR.

FIG. 2 shows that the 1E6 hybridoma clone competes with GS-1811 benchmark in binding to CCR8 expressing cells.

FIGS. 3A to 3C show that anti-CCR8 antibodies derived from chimera or humanization of hybridoma clone 1E6 bind specifically to human CCR8 and not human CCR4 or mouse CCR8.

FIGS. 4A and 4B show the human CCR8 binding potency of the anti-CCR8 antibodies.

FIGS. 5A and 5B show the antagonist activity of the anti-CCR8 antibodies.

FIGS. 6A to 6C show the antibody-dependent cell cytotoxicity (ADCC) activity of the anti-CCR8 antibodies using human peripheral blood mononuclear cell (PBMC) against CHO-K1 cells overexpressing human CCR8.

FIG. 7 is a graph that shows the thermal stability of the anti-CCR8 antibodies.

FIGS. 8A to 8D shows the study design and results for the in vivo efficacy study using anti-CCR8 antibody SD-171467-afuc as a monotherapy. FIGS. 8B and 8C shows tumor volume and percent tumor volume change after drug treatment. FIG. 8D shows the changes in mouse body weights from drug administrations.

FIGS. 9A to 9C show the results of the pharmacokinetic studies. FIG. 9A shows the pharmacokinetic study design for SD-171467-afuc. FIG. 9B shows the pharmacokinetic profile of SD-171467-afuc. FIG. 9C summarizes the calculated pharmacokinetic parameters.

FIGS. 10A to 10F show the study design and results for the in vivo efficacy study using SD-171467-afuc and anti-PD1 antibody as combination therapy and the mechanism of action study for CCR8+ Treg depletion. FIG. 10A shows the in vivo study design for SD-171467-afuc combination treatment with anti-PD1 antibody and CCR8+ Treg depletion. FIGS. 10B and 10C show tumor volume and percent tumor volume change after drug treatment. FIG. 10D shows the changes in mouse body weights from drug administrations. FIGS. 10E and 10F show Treg-CCR8+ levels in tumor and spleen after PBS or SD-171467-afuc treatment.

### DETAILED DESCRIPTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

It should be understood that, unless clearly indicated, in any method described or disclosed herein that includes more than one act, the order of the acts is not necessarily limited to the order in which the acts of the method are recited, but the disclosure encompasses exemplary embodiments in which the order of the acts is so limited.

The present invention is directed to novel anti-CCR8 antibodies and antigen binding fragments thereof for use in binding to human CCR8 without binding to other members of the chemokine receptor family, including the closely related CCR4.

Chemokine (C—C motif) receptor 8, also known as CCR8, is a protein which in humans is encoded by the CCR8 gene, also designated CDw198 (cluster of differentiation w198). CCR8 is a member of the beta chemokine receptor family, which is a seven transmembrane G protein-coupled receptor (GPCR) protein. The ligand for CCR8 is CCL1. Human CCR8 is UniProt P51685, RefSeq (mRNA) NM_005201 and RefSeq (protein) NP_005192.

CCR8 is expressed primarily on regulatory T cells (Treg) and is important for CCR8+ regulatory T cell (Treg)-mediated immunosuppression. Recent studies have shown that CCR8 is upregulated in human tumor-resident Tregs and CCR8⁺ myeloid cells of cancer patients, and both cells are expanded in patients with cancer.

The antibodies of the present invention can be used to treat cancer by eliminating Tregs that inhibit anti-tumor cell T cells, to treat or inhibit cancer. In some cases, the cancer cells in the patient express or overexpress CCR8. Cancer that can be treated by eliminating Tregs (sometimes referred to as tumor infiltrating lymphocytes (TILs)) include, e.g., gastric, pancreatic, esophageal, ovarian, and lung tumors.

The term “antibody” as used herein throughout is used in the broadest sense and includes a monoclonal antibody, polyclonal antibody, human antibody, humanized antibody, non-human antibody, chimeric antibody, a monovalent antibody, an antibody fragment, and a tandem scFv-Fc antibody.

Antibody fragments of the disclosure retain CCR8 antigen binding specificity. Antibody fragments include antigen-binding fragments (Fab), variable fragments (Fv) containing VH and VL sequences, single chain variable fragments (scFv) containing VH and VL sequences linked together in one chain, single chain antibody fragments (scAb) or other antibody variable region fragments, such as retaining antigen binding specificity.

Tandem scFv-Fc antibodies of the disclosure are composed two or more scFv binding sites in tandem on each antibody arm, optionally linked by a linker, optionally a flexible linker, giving rise to a total of four or more scFv binding sites in a single scFv-Fc formatted antibody.

The term “meso scale-molecule (MEM)” as used herein throughout includes engineered peptides and polypeptides between about 1 kDa and about 10 kDa. The term “MEM-nanoparticle” as used herein throughout includes MEMS which have been conjugated to a nanoparticle (e.g., ferritin nanoparticle).

As used herein, a “subject” may be a mammalian subject. Mammalian subjects include, humans, non-human primates, rodents, (e.g., rats, mice), lagomorphs (e.g., rabbits), ungulates (e.g., cows, sheep, pigs, horses, goats, and the like), etc. In some embodiments, the subject is a human. In some embodiments, the subject is a non-human primate, for example a cynomolgus monkey. In some embodiments, the subject is a companion animal (e.g., cats, dogs).

All publications, patents, and patent applications mentioned in this specification are herein incorporated by reference to the same extent as if each individual publication, patent, or patent application was specifically and individually indicated to be incorporated by reference.

Antibodies.

As used herein, the term “antibody” refers to an intact antibody or a binding fragment thereof that binds specifically to a target antigen. Binding fragments are produced by recombinant DNA techniques, or by enzymatic or chemical cleavage of intact antibodies. Binding fragments include Fab, Fab′, F(ab′)2, Fv, and single-chain variable fragment (scFv) antibodies. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay). The term “antibody” is used in the broadest sense, and specifically covers monoclonal antibodies (including full-length antibodies or other bivalent, Fc-region containing antibodies such as bivalent scFv Fc-fusion antibodies), polyclonal antibodies, multispecific antibodies (e.g., bispecific antibodies), and antibody fragments (e.g., Fab, Fab′, F(ab′)2, Fv, scFv) so long as they exhibit the desired biological activity. Antibodies (Abs) and immunoglobulins (Igs) are glycoproteins having the same structural characteristics. The present invention includes monoclonal antibodies (and binding fragments thereof) that are completely recombinant, in other words, where the complementarity determining regions (CDRs) are genetically spliced into a human antibody backbone, often referred to as veneering an antibody. Thus, in certain aspects, the monoclonal antibody is a fully synthesized antibody. In certain embodiments, the monoclonal antibodies (and binding fragments thereof) can be made in bacterial, fungal, mammalian, insect, or plant cells.

As used herein, the term “antibody fragment” refers to a portion of a full-length antibody, generally the antigen-binding or variable region, and include Fab, Fab′, F(ab′)2, Fv, and scFv fragments. Papain digestion of antibodies produces two identical antigen-binding fragments, called the Fab fragment, each with a single antigen-binding site, and a residual “Fc” fragment, so-called for its ability to crystallize readily. Pepsin treatment yields an F(ab′)2 fragment that has two antigen-binding fragments which are capable of cross-linking antigen, and a residual other fragment (which is termed pFc′). As used herein, “functional fragment” with respect to antibodies, refers to Fv, F(ab) and F(ab′)2 fragments.

As used herein, the “Fv” fragment is the minimum antibody fragment that contains a complete antigen recognition and binding site. This region consists of a dimer of one heavy and one light chain variable domain in a tight, non-covalent association (VH-VL dimer). It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment, also designated as F(ab), also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)2 pepsin digestion product. Additional chemical couplings of antibody fragments are known to those of ordinary skill in the art.

Native antibodies and immunoglobulins are usually heterotetrameric glycoproteins of about 150,000 daltons, composed of two identical light (L) chains and two identical heavy (H) chains. Each light chain is linked to a heavy chain by at least one covalent disulfide bond, however, the number of disulfide linkages varies between the heavy chains of different immunoglobulin isotypes. Each heavy and light chain also has regularly spaced intrachain disulfide bridges. Each heavy chain has at one end a variable domain (VH) followed by the constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82 4592-4596 (1985), relevant portions incorporated herein by reference.

As used herein, an “isolated” antibody is one that has been identified and separated and/or recovered from a component of the environment in which it was produced. Contaminant components of its production environment are materials, which would interfere with diagnostic or therapeutic uses for the antibody, and may include enzymes, hormones, and other proteinaceous or nonproteinaceous solutes. In certain embodiments, the antibody will be purified as measurable by at least three different methods: 1) to greater than 50% by weight of antibody as determined by the Lowry method, such as more than 75% by weight, or more than 85% by weight, or more than 95% by weight, or more than 99% by weight; 2) to a degree sufficient to obtain at least 10 residues of N-terminal or internal amino acid sequence by use of a spinning cup sequentator, such as at least 15 residues of sequence; or 3) to homogeneity by SDS-PAGE under reducing or non-reducing conditions using Coomasie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, the terms “antibody mutant” or “antibody variant” refer to an amino acid sequence variant of an antibody wherein one or more of the amino acid residues have been modified. Such mutants necessarily have less than 100% sequence identity or similarity with the amino acid sequence having at least 75% amino acid sequence identity or similarity with the amino acid sequence of either the heavy or light chain variable domain of the antibody, such as at least 80%, or at least 85%, or at least 90%, or at least 95, 96, 97, 98, or 99%.

As used herein, the term “variable” in the context of the variable domain of antibodies, refers to the fact that certain portions of the variable domains differ extensively in sequence among antibodies and are used in the binding and specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains. There are at least two techniques for determining CDRs: (1) an approach based on cross-species sequence variability (i.e., Kabat et al., Sequences of Proteins of Immunological Interest (National Institute of Health, Bethesda, Md. 1987); and (2) an approach based on crystallographic studies of antigen-antibody complexes (Chothia, C. et al. (1989), Nature 342: 877), or both, that is Chothia plus Kabat. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al.). The constant domains are not involved directly in binding an antibody to its cognate antigen but exhibit various effector function, such as participation of the antibody in antibody-dependent cellular toxicity.

The light chains of antibodies (immunoglobulin) from any vertebrate species can be assigned to one of two clearly distinct types, called kappa and lambda, based on the amino sequences of their constant domain. Depending on the amino acid sequences of the constant domain of their heavy chains, “immunoglobulins” can be assigned to different classes. There are at least five (5) major classes of immunoglobulins: IgA, IgD, IgE, IgG, and IgM, and several of these may be further divided into subclasses (isotypes), e.g., IgG-1, IgG-2, IgG-3, and IgG4; IgA-1 and IgA-2. The subunit structures and three-dimensional configurations of different classes of immunoglobulins are well known.

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed and claimed invention may be made by the hybridoma method first described by Kohler and Milstein, Nature 256, 495 (1975), relevant portions incorporated herein by reference.

All monoclonal antibodies used in accordance with the presently disclosed and claimed invention will be either (1) the result of a deliberate immunization protocol, as described in more detail hereinbelow; or (2) the result of an immune response that results in the production of antibodies naturally in the course of a disease or cancer.

The uses of the monoclonal antibodies of the presently disclosed and claimed invention may require administration of such or similar monoclonal antibody to a subject, such as a human. However, when the monoclonal antibodies are produced in a non-human animal, such as a rodent or chicken, administration of such antibodies to a human patient will normally elicit an immune response, wherein the immune response is directed towards the antibodies themselves. Such reactions limit the duration and effectiveness of such a therapy. In order to overcome such problem, the monoclonal antibodies of the presently disclosed and claimed invention can be “humanized”, that is, the antibodies are engineered such that antigenic portions thereof are removed and like portions of a human antibody are substituted therefore, while the antibodies' affinity for CCR8. This engineering may only involve a few amino acids, or may include entire framework regions of the antibody, leaving only the complementarity determining regions of the antibody intact. Several methods of humanizing antibodies are known in the art and are disclosed in U.S. Pat. No. 6,180,370, issued to Queen et al on Jan. 30, 2001; U.S. Pat. No. 6,054,927, issued to Brickell on Apr. 25, 2000; U.S. Pat. No. 5,869,619, issued to Studnicka on Feb. 9, 1999; U.S. Pat. No. 5,861,155, issued to Lin on Jan. 19, 1999; U.S. Pat. No. 5,712,120, issued to Rodriquez et al on Jan. 27, 1998; and U.S. Pat. No. 4,816,567, issued to Cabilly et al on Mar. 28, 1989, relevant portions incorporated herein by reference.

Humanized forms of antibodies are chimeric immunoglobulins, immunoglobulin chains or fragments thereof (such as Fab, Fab′, F(ab′)2, Fv, scFv or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., 1986; Riechmann et al., 1988; Verhoeyen et al., 1988), by substituting nonhuman (i.e., rodent, chicken) CDRs or CDR sequences for the corresponding sequences of a human antibody, see, e.g., U.S. Pat. No. 5,225,539. In some instances, Fv framework residues of the human immunoglobulin are replaced by corresponding non-human residues from the donor antibody. Humanized antibodies can also comprise residues that are found neither in the recipient antibody nor in the imported CDR or framework sequences. In general, the humanized antibody will comprise substantially all of, at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin.

The presently disclosed and claimed invention further includes the use of fully human monoclonal antibodies against CCR8. Fully human antibodies essentially relate to antibody molecules in which the entire sequence of both the light chain and the heavy chain, including the CDRs, arise from human genes. Such antibodies are termed “human antibodies” or “fully human antibodies” herein. Human monoclonal antibodies can be prepared by, e.g., the trioma technique; the human B-cell hybridoma technique (see Kozbor, et al., Hybridoma, 2:7 (1983)) and the EBV hybridoma technique to produce human monoclonal antibodies (see Cole, et al., PNAS 82:859 (1985)), or as taught herein. Human monoclonal antibodies may be utilized in the practice of the presently disclosed and claimed invention and may be produced by using human hybridomas (see Cote, et al., PNAS 80:2026 (1983)) or by transforming human B-cells with Epstein Barr Virus in vitro (see Cole, et al., 1985), relevant portions incorporated herein by reference.

In addition, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example but not by way of limitation, in U.S. Pat. Nos. 5,545,807; 5,545,806; 5,569,825; 5,625,126; 5,633,425; 5,661,016, and in Marks et al., J Biol. Chem. 267:16007, (1992); Lonberg et al., Nature, 368:856 (1994); Morrison, 1994; Fishwild et al., Nature Biotechnol. 14:845 (1996); Neuberger, Nat. Biotechnol. 14:826 (1996); and Lonberg and Huszar, Int Rev Immunol. 13:65 (1995), relevant portions incorporated herein by reference.

A method for producing an antibody of interest, such as a human antibody, is disclosed in U.S. Pat. No. 5,916,771, issued to Hori et al. on Jun. 29, 1999, and incorporated herein by reference. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

As used herein, the term “treatment” refers to both therapeutic treatment and prophylactic or preventative measures. Those in need of treatment include those already with the disorder as well as those in which the disorder is to be prevented.

As used herein, the term “disorder” refers to any condition that would benefit from treatment with the polypeptide. This includes chronic and acute disorders or diseases including those infectious or pathological conditions that predispose the mammal to the disorder in question.

An antibody or antibody fragment can be generated with an engineered sequence or glycosylation state to confer preferred levels of activity in antibody-dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

Alternatively, or additionally, it may be useful to combine amino acid modifications with one or more further amino acid modifications that alter complement component Clq binding and/or the complement-dependent cytotoxicity (CDC) function of the Fc region of an IL-23p19 binding molecule. The binding polypeptide of particular interest may be one that binds to Clq and displays complement-dependent cytotoxicity. Polypeptides with pre-existing Clq binding activity, optionally further having the ability to mediate CDC may be modified such that one or both of these activities are enhanced. Amino acid modifications that alter Clq and/or modify its complement-dependent cytotoxicity function are described, for example, in WO/0042072, which is hereby incorporated by reference.

An Fc region of an antibody can be designed to alter the effector function, e.g., by modifying Clq binding and/or FcγR binding and thereby changing complement-dependent cytotoxicity (CDC) activity and/or antibody-dependent cell-mediated cytotoxicity (ADCC) activity. These “effector functions” are responsible for activating or diminishing a biological activity (e.g., in a subject). Examples of effector functions include, but are not limited to: Clq binding; CDC; Fc receptor binding; ADCC; phagocytosis; down-regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved Clq binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen-binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine, and glycine. However, other residues can function as well. Phage display can be used to rapidly select tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) is displayed on filamentous phage and subjected to affinity selection with hapten. The population of selected variants exhibited significant increases in binding activity but retained considerable sequence diversity. Sequence analysis revealed a conserved proline in the linker two residues after the VH C terminus and an abundance of arginines and prolines at other positions as the only common features of the selected tethers. In certain embodiments, the antibody fragments are further modified to increase their serum half-life by using modified Fc regions or mutations to the various constant regions, as are known in the art.

In certain embodiments, the antibodies of the present invention are formulated for administration to humans. For example, the antibodies of the present invention can be included in a pharmaceutical composition formulated for an administration that is: intranasal, intrapulmonary, intrabronchial, intravenous, oral, intraadiposal, intraarterial, intraarticular, intracranial, intradermal, intralesional, intramuscular, intrapericardial, intraperitoneal, intrapleural, intravesicular, local, mucosal, parenteral, enteral, subcutaneous, sublingual, topical, transbuccal, transdermal, via inhalation, via injection, in creams, in lipid compositions, via a catheter, via a lavage, via continuous infusion, via infusion, via local delivery, or via localized perfusion, and wherein the composition is a serum, drop, gel, ointment, spray, reservoir, or mist.

As used herein, the term “antigen” refers to a molecule containing one or more epitopes (either linear, conformational or both) that will stimulate a host's immune-system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 15 amino acids. The term includes polypeptides, which include modifications, such as deletions, additions and substitutions (generally conservative in nature) as compared to a native sequence, so long as the protein maintains the ability to elicit an immunological response, as defined herein. These modifications may be deliberate, as through site-directed mutagenesis, or may be accidental, such as through mutations of hosts, which produce the antigens.

As used herein, the term “epitope” refers to a specific amino acid sequence or molecule (such as a carbohydrate, small molecule, lipid, etc.) that when present in the proper conformation, provides a reactive site for an antibody (e.g., B cell epitope) or in the case of a peptide to a T cell receptor (e.g., T cell epitope).

Portions of a given polypeptide that include a B-cell epitope can be identified using any number of epitope mapping techniques that are known in the art. (See, e.g., Epitope Mapping Protocols in Methods in Molecular Biology, Vol. 66, Glenn E. Morris, Ed., 1996, Humana Press, Totowa, N.J.). For example, linear epitopes can be determined by, e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad Sci. USA 81:3998-4002; Geysen et al. (1986) Molec. Immunol. 23:709-715.

As used herein, the term “substantially purified” refers to isolation of a substance (compound, polynucleotide, protein, polypeptide, polypeptide composition) such that the substance comprises the majority percent of the sample in which it resides. Typically, in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

As used herein, the term “treatment” refers to any of (i) the prevention of infection or reinfection, as in a traditional vaccine, (ii) the reduction or elimination of symptoms, and (iii) the substantial or complete elimination of the cancers in question.

The practice of the present invention employs, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18^{th} Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2^{nd} Edition, 1989); Short Protocols in Molecular Biology, 4^{th} ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2^{nd} ed. (Newton & Graham eds., 1997, Springer Verlag); Fundamental Virology, Second Edition (Fields & Knipe eds., 1991, Raven Press, New York), relevant portion incorporated herein by reference.

Conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

Methods of expressing DNA sequences having eukaryotic or viral sequences in prokaryotes are well known in the art. Non-limiting examples of suitable host cells include bacteria, archea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, SF9 cells, C129 cells, 293 cells, Neurospora, and immortalized mammalian myeloid and lymphoid cell lines. Techniques for the propagation of mammalian cells in culture are well-known (see, Jakoby and Pastan (eds), 1979, Cell Culture. Methods in Enzymology, volume 58, Academic Press, Inc., Harcourt Brace Jovanovich, N.Y.). Examples of commonly used mammalian host cell lines are VERO and HeLa cells, CHO cells, and WI38, BHK, and COS cell lines, although cell lines may be used, such as cells designed to provide higher expression desirable glycosylation patterns, or other features. As discussed above, techniques for the transformation of yeast cells, such as polyethylene glycol transformation, protoplast transformation and gene guns are also known in the art (see Gietz and Woods, Meth Enzymol 350: 87-96, 2002).

Transformation of a host cell with recombinant DNA can be carried out by conventional techniques as are well known to those skilled in the art. Where the host is prokaryotic, such as, but not limited to, E. coli, competent cells which are capable of DNA uptake can be prepared from cells harvested after exponential growth phase and subsequently treated by the CaCl2 method using procedures well known in the art. Alternatively, MgCl2 or RbCl can be used. Transformation can also be performed after forming a protoplast of the host cell if desired, or by electroporation.

When the host is a eukaryote, such methods of transfection of DNA as calcium phosphate coprecipitates, conventional mechanical procedures such as microinjection, electroporation, insertion of a plasmid encased in liposomes, or virus vectors can be used. Eukaryotic cells can also be co-transformed with polynucleotide sequences encoding the antibody(ies) or portion(s) thereof, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982).

The antibody(ies) or portion(s) thereof and variants also can be produced in plants. For example, polypeptides can be expressed in plants using the IBIOLAUNCH™ gene expression platform (iBio, Inc., Newark, DE) as described in, for example, U.S. Pat. No. 7,491,509 (which is incorporated herein by reference in its entirety). The IBIOLAUNCH™ platform can be used to produce high levels of target proteins in non-transgenic plants. This platform can have benefits over methods utilizing animal cells, or microbes, and over systems that require transgenic plants.

Therapeutic Methods and Pharmaceutical Compositions.

The antibody or portions thereof disclosed herein, or nucleic acids encoding the antibody or portions thereof, can be used to treat cancer. In several examples, the antibody or portions thereof, or nucleic acid encoding these polypeptides are of use to decrease cancer, such as in a subject. Thus, in several embodiments, the methods include administering to a subject a therapeutically effective amount of one or more of the antibody or portions thereof disclosed herein, or polynucleotides encoding these polypeptides, in order to decrease cancer. However, any of the antibody or portions thereof disclosed herein can be used to decrease cancer. In some embodiments, the peptides can be administered as a unit dose. In some embodiments, the polypeptides are administered as multimers.

A therapeutically effect amount of the antibody(ies) or portion(s) thereof, or polynucleotide encoding the peptide can be administered in the pharmaceutically acceptable carrier. Pharmacologically acceptable carriers (e.g., physiologically or pharmaceutically acceptable carriers) are well known in the art, and include, but are not limited to buffered solutions as a physiological pH (e.g., from a pH of about 7.0 to about 8.0, or at a pH of about 7.4). One specific, non-limiting example of a physiologically compatible buffered solution is phosphate buffered saline. Other pharmacologically acceptable carriers include penetrants, which are particularly suitable for pharmaceutical formulations that are intended to be topically applied (for example in the application of surgical wounds to promote healing).

The pharmacological compositions disclosed herein facilitate the use of at least one the antibody(ies) or portion(s) thereof, or polynucleotide encoding the antibody(ies) or portion(s) thereof, either in vivo or ex vivo, to decrease cancer. Such a composition can be suitable for delivery of the active ingredient to any suitable subject, and can be manufactured in a manner that is itself known, e.g., by means of conventional mixing, dissolving, granulating, emulsifying, encapsulating, entrapping or lyophilizing processes. Pharmacological compositions can be formulated in a conventional manner using one or more pharmacologically (e.g., physiologically or pharmaceutically) acceptable carriers, as well as optional auxiliaries that facilitate processing of the active compounds into preparations which can be used pharmaceutically. Proper formulation is dependent upon the route of administration chosen. Thus, for injection, the active ingredient can be formulated in aqueous solutions. For transmucosal administration, penetrants appropriate to the barrier to be permeated are used in the formulation. Such penetrants are generally known in the art.

For oral administration, a therapeutically effective amount of at least one antibody(ies) or portion(s) thereof, or a nucleic acid encoding the peptide, can be combined with carriers suitable for incorporation into tablets, pills, capsules, liquids, gels, syrups, slurries, suspensions and the like.

Protein drugs are subject to protease-mediated degradation in the digestive tract through the action of enzymes such as trypsin, chymotrypsin, and brush border peptidases, such that oral administration of large protein molecules often does not result in the intended therapeutic effect (Soltero and Ekwruibe, 2001 Innovations in Pharmaceutical Technology, 1:106-110).

In some embodiments, for parenteral administration, a therapeutically effective amount of the antibody(ies) or portion(s) thereof, or a nucleic acid encoding the peptide, can be administered by injection, such as by bolus injection or continuous infusion. Such compositions can take such forms as suspensions, solutions or emulsions in oily or aqueous vehicles, and can contain formulatory agents such as suspending, stabilizing and/or dispersing agents. Other pharmacological excipients are known in the art.

Optionally, the antibody(ies) or portion(s) thereof, or polynucleotide encoding the peptide can be contained within or conjugated with a heterologous protein, hydrocarbon or lipid, whether for in vitro or in vivo administration. Co-administration can be such that the antibody(ies) or portion(s) thereof, or polynucleotide encoding the peptide is administered before, at substantially the same time as, or after the protein, hydrocarbon, or lipid. In some embodiments, the antibody(ies) or portion(s) thereof, or polynucleotide encoding the peptide is administered at substantially the same time, as the protein, hydrocarbon, or lipid.

Other delivery systems can include time-release, delayed release or sustained release delivery systems. Such systems can avoid repeated administrations of the compositions of the invention described above, increasing convenience to the subject and the physician. Many types of release delivery systems are available and known to those of ordinary skill in the art. They include polymer based systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems, such as lipids including sterols such as cholesterol, cholesterol esters and fatty acids or neutral fats such as mono- di- and tri-glycerides; hydrogel release systems; silastic systems; peptide based systems; wax coatings; compressed tablets using conventional binders and excipients; partially fused implants; and the like. Specific examples include, but are not limited to: (a) erosional systems in which the antibody(ies) or portion(s) thereof, or polynucleotide encoding the peptide is contained in a form within a matrix such as those described in U.S. Pat. Nos. 4,452,775; 4,667,014; 4,748,034; 5,239,660; and 6,218,371 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

The therapeutically effective amount of the antibody(ies) or portion(s) thereof, or polynucleotide encoding the peptide will be dependent on the antibody(ies) or portion(s) thereof, or polynucleotide encoding the peptide that is utilized, the subject being treated, the severity and type of the affliction, and the manner of administration. For example, a therapeutically effective amount of a polynucleotide encoding the peptide can vary from about 0.01 μg per kilogram (kg) body weight to about 1 g per kg body weight, such as about 1 μg to about 5 mg per kg body weight, or about 5 μg to about 1 mg per kg body weight. The exact dose is readily determined by one of skill in the art based on the potency of the specific compound the age, weight, sex and physiological condition of the subject.

With regard to the administration of nucleic acids, one approach to administration of nucleic acids is direct treatment with plasmid DNA, such as with a mammalian expression plasmid. As described above, the nucleotide sequence encoding the antibody(ies) or portion(s) thereof can be placed under the control of a promoter to increase expression of the molecule.

When a viral vector is utilized for administration in vivo, it is desirable to provide the recipient with a dosage of each recombinant virus in the composition in the range of from about 10⁵ to about 10¹⁰ plaque forming units/mg mammal, although a lower or higher dose can be administered. The composition of recombinant viral vectors can be introduced into a mammal either prior to any evidence of a cancer, or to mediate regression of the disease in a mammal afflicted with the cancer. Examples of methods for administering the composition into mammals include, but are not limited to, exposure of cells to the recombinant virus ex vivo, or injection of the composition into the affected tissue or intravenous, subcutaneous, intradermal or intramuscular administration of the virus. Alternatively, the recombinant viral vector or combination of recombinant viral vectors may be administered locally by direct injection into the cancerous lesion in a pharmaceutically acceptable carrier. Generally, the quantity of recombinant viral vector, carrying the nucleic acid sequence of one or more antibody or portions thereof to be administered is based on the titer of virus particles. An exemplary range of the immunogen to be administered is 10⁵ to 10¹⁰ virus particles per mammal, such as a human.

In one specific, non-limiting example, a pharmaceutical composition for intravenous administration would include about 0.1 μg to 10 mg of the antibody(ies) or portion(s) thereof per patient per day. Dosages from 0.1 up to about 100 mg per patient per day can be used, particularly if the agent is administered to a secluded site and not into the circulatory or lymph system, such as into a body cavity or into a lumen of an organ. Actual methods for preparing administrable compositions will be known or apparent to those skilled in the art and are described in more detail in such publications as Remington's Pharmaceutical Sciences, 19th Ed., Mack Publishing Company, Easton, Pa., 1995.

Single or multiple administrations of the compositions are administered depending on the dosage and frequency as required and tolerated by the subject. In some embodiments, the dosage is administered once as a bolus, but in another embodiment can be applied periodically until a therapeutic result is achieved. Generally, the dose is sufficient to treat or ameliorate symptoms or signs of disease without producing unacceptable toxicity to the subject. Systemic or local administration can be utilized.

In a further method, an additional agent is administered. In one example, this administration is sequential. In other examples, the additional agent is administered simultaneously with the antibody(ies) or portion(s) thereof.

The invention will be further described in the following examples, which do not limit the scope of the invention described in the claims.

Provided herein are antibodies that bind to CCR8. These antibodies are referred to herein as anti-CCR8 antibodies. A number of discovery strategies have been employed to obtain the exemplary antibodies of the disclosure, further discussed below.

In some embodiments, the MEM-nanoparticles along with full length anti-CCR8 are used to immunize a subject in order to produce antibodies specific to the MEM epitope. Monoclonal hybridomas are then created to produce epitope specific anti-CCR8 antibodies. In other embodiments, mouse serum is collected and used to produce in vitro scFv libraries. Followed by phage panning against full length anti-CCR8 and the MEM-nanoparticle to isolate epitope specific clones.

In some embodiments, anti-CCR8 antibodies have a half maximal effective concentration (EC₅₀) to CCR8 of about 1.6, 1.8, 1.9, 2.0, 2.2, 2.3, 2.6, or 2.7 nM.

The skilled artisan will recognize that binding specificity may be determined through a series of competition binding paradigms, in which a desired antibody demonstrates its ability to prevent binding of a known reference antibody to its target epitope at varying concentrations. In some embodiments, the reference anti-CCR8 antibody is GS-1811.

In some embodiments, the anti-CCR8 antibody is a full-length antibody (referring to an antibody with two heavy and two light chains attached to the Fc domain, giving a ‘Y’ shape). In some embodiments the Fc domain (or simply referred to as an Fc) is a human Fc domain. In some embodiments, the Fc domain of an anti-CCR8 antibody is from a human IgG1, human IgG2, human IgG3, or human IgG4.

Exemplary Anti-CCR8 Antibodies—CDR Sequences.

Provided herein are sequences for exemplary anti-CCR8 antibodies of the disclosure. Included are complementarity determining region (CDR) sequences and the variable heavy and light domain sequences (VH, VL) that constitute the anti-CCR8 antigen binding domains of the disclosure. The discovery of these antibodies is detailed in the Examples section.

As referred below, a light chain variable (VL) domain CDR1 region is referred to as CDR-L1; a VL CDR2 region is referred to as CDR-L2; a VL CDR3 region is referred to as CDR-L3; a heavy chain variable (VH) domain CDR1 region is referred to as CDR-H1; a VH CDR2 region is referred to as CDR-H2; and a VH CDR3 region is referred to as CDR-H3. Table 1 provides exemplary CDR combinations of antibodies of the disclosure.

TABLE 1
Antibody CDRs

| | Antibody | | |
|---|---|---|---|
| | CDR1 | CDR2 | CDR3 |
| 1E6H2K - HC | GFTFNTNA | IRSKSN | VRGKDTSGS |
| | | NYAT | YYAMDY |
| SEQ ID NO: | 1 | 2 | 3 |
| 1E6H2K - LC | KSLLHSNG | RMS | MQHLEYPFT |
| | NTY | | |
| SEQ ID NO: | 4 | 5 | 6 |

Tables 2A and 2B shows the heavy chain and light chain anti-CCR8 humanized antibody amino acid sequences, respectively. The CDRs for every heavy chain and light chain are in Table 1.

TABLE 2A
Anti-CCR8 humanized antibody amino acid sequences -
heavy chains.

| Antibody | | SEQ ID |
|---|---|---|
| ID | VH Seq | NO: |
| SD-356253_VH | QVQLVETGGGLVQPKGSLKLSCAASGFTFNTNAMNWVRQAPG | 7 |
| | KGLEWLARIRSKSNNYATYYADSVKDRFTISRDDSQSMLYLQ | |
| | MKNLKSEDTAMYYCVRGKDTSGSYYAMDYWGQGTSVTVSS | |
| SD-171467_VH | QVQLVESGGGLVQPGGSLKLSCAASGFTFNTNAMNWVRQASG | 8 |
| | KGLEWVGRIRSKSNNYATYYADSVKDRFTISRDDSKNTLYLQ | |
| | MNSLKTEDTAVYYCVRGKDTSGSYYAMDYWGQGTTVTVSS | |
| SD-962141_VH | EVQLVESGGGLVQPGGSLKLSCAASGFTFNTNAMNWVRQAPG | 9 |
| | KGLEWVGRIRSKSNNYATYYADSVKDRFTISRDDSKSTLYLQ | |
| | MNSLKTEDTAVYYCVRGKDTSGSYYAMDYWGQGTLVTVSS | |
| SD-611534_VH | EVQLVESGGGLVQPGGSLKLSCAASGFTFSTNAMNWVRQAPG | 10 |
| | KGLEWVGRIRSKSNNYATYYADSVKDRFTISRDDSKNTLYLQ | |
| | MNSLKTEDTAVYYCVRGKDTSGSYYAMDYWGQGTLVTVSS | |
| SD-580600_VH | QVQLVESGGGLVQPGGSLKLSCAASGFTFNTNAMNWVRQASG | 11 |
| | KGLEWVGRIRSKSNNYATYYADSVKDRFTISRDDSKNTLYLQ | |
| | MNSLKTEDTAVYYCVRGKDTSGSYYAMDYWGQGTTVTVSS | |
| SD-388950_VH | QVQLVESGGGLVQPGGSLKLSCAASGFTFNTNAMNWVRQASG | 12 |
| | KGLEWVGRIRSKSNNYATYYADSVKDRFTISRDDSQSMLYLQ | |
| | MNSLKTEDTAVYYCVRGKDTSGSYYAMDYWGQGTTVTVSS | |
| SD-117248_VH | QVQLVESGGGLVQPGGSLKLSCAASGFTFNTNAMNWVRQASG | 13 |
| | KGLEWVGRIRSKSNNYATYYADSVKDRFTISRDDSKNTLYLQ | |
| | MNSLKTEDTAVYYCVRGKDTSGSYYAMDYWGQGTTVTVSS | |
| SD-166154_VH | EVQLVESGGGLVQPGGSLKLSCAASGFTFSTNAMNWVRQASG | 14 |
| | KGLEWVGRIRSKSNNYATYYADSVKDRFTISRDDSKNTAYLQ | |
| | MNSLKTEDTAVYYCTRGKDTSGSYYAMDYWGQGTMVTVSS | |
| SD-969683_VH | EVQLVESGGGLVQPGGSLKLSCAASGFTFSTNAMNWVRQASG | 15 |
| | KGLEWVGRIRSKSNNYATYYADSVKDRFTISRDDSKNTAYLQ | |
| | MNSLKTEDTAVYYCTRGKDTSGSYYAMDYWGQGTMVTVSS | |

TABLE 2B
Anti-CCR8 humanized antibody amino acid sequences -
light chains.

| | | SEQ ID |
|---|---|---|
| Seq ID | VL Seq | NO: |
| SD- | DIVMTQAAPSVPVTPGESVSISCRSSKSLLHSNGNTYLYWFLQRP | 16 |
| 356253_VL | GQSPQLLIYRMSNLASGVPDRFSGSGSGTAFTLRISRVEAEDVGV | |
| | YYCMQHLEYPFTFGSGTKLEIR | |
| SD- | DIVMTQSPLSLPVTPGEPASISCRSSKSLLHSNGNTYLYWFLQKP | 17 |
| 171467_VL | GQSPQLLIYRMSNLASGVPDRFSGSGSGTDFTLKISRVEAEDVGV | |
| | YYCMQHLEYPFTFGGGTKVEIK | |
| SD- | DIVMTQSPLSLPVTPGEPASISCRSSKSLLHSNGNTYLYWFLQKP | 18 |
| 962141_VL | GQSPQLLIYRMSNLASGVPDRESGSGSGTDFTLKISRVEAEDVGV | |
| | YYCMQHLEYPFTFGGGTKVEIK | |
| SD- | DIVMTQSPLSLPVTPGEPASISCRSSKSLLHSNGNTYLYWFLQKP | 19 |
| 611534_VL | GQSPQLLIYRMSNLASGVPDRESGSGSGTDFTLKISRVEAEDVGV | |
| | YYCMQHLEYPFTFGGGTKVEIK | |
| SD- | DIVMTQSALSLPVTPGEPASISCRSSKSLLHSNGNTYLYWFLQKP | 20 |
| 580600_VL | GQSPQLLIYRMSNLASGVPDRFSGSGSGTDFTLKISRVEAEDVGV | |
| | YYCMQHLEYPFTFGGGTKVEIK | |
| SD- | DIVMTQSALSLPVTPGEPASISCRSSKSLLHSNGNTYLYWFLQKP | 21 |
| 388950_VL | GQSPQLLIYRMSNLASGVPDRFSGSGSGTDFTLKISRVEAEDVGV | |
| | YYCMQHLEYPFTFGGGTKVEIK | |
| SD- | DIVMTQSPLSLPVTPGEPASISCRSSKSLLHSNGNTYLYWFLQKP | 22 |
| 117248_VL | GQSPQLLIYRMSNLASGVPDRFSGSGSGTAFTLKISRVEAEDVGV | |
| | YYCMQHLEYPFTFGGGTKVEIK | |
| SD- | DIVMTQSPLSLPVTPGEPASISCRSSKSLLHSNGNTYLYWFLQKP | 23 |
| 166154_VL | GQSPQLLIYRMSNLASGVPDRESGSGSGTAFTLKISRVEAEDVGV | |
| | YYCMQHLEYPFTFGGGTKVEIK | |
| SD- | DIVMTQSPLSLPVTPGESVSISCRSSKSLLHSNGNTYLYWFLQKP | 24 |
| 969683_VL | GQSPQLLIYRMSNLASGVPDRFSGSGSGTDFTLRISRVEAEDVGV | |
| | YYCMQHLEYPFTFGGGTKVEIK | |

TABLE 3A
Anti-CCR8 humanized antibody nucleic acid sequences -
heavy chains.

| | | SEQ ID |
|---|---|---|
| Seq ID | VH Seq | NO: |
| SD- | CAAGTGCAGCTCGTGGAAACTGGCGGCGGACTGGTGCAGCCG | 25 |
| 356253_VH | AAGGGTAGCCTGAAGCTGTCCTGTGCGGCCTCCGGTTTCACT | |
| | TTTAACACCAACGCCATGAATTGGGTCCGCCAGGCACCCGGG | |
| | AAAGGGCTGGAATGGCTCGCGAGAATCCGGTCCAAGTCGAAC | |
| | AACTACGCCACCTACTACGCTGACTCGGTCAAGGACAGGTTC | |
| | ACGATTTCCCGCGACGATTCACAGAGCATGCTGTACCTTCAA | |
| | ATGAAGAACTTGAAGTCAGAGGATACCGCCATGTACTATTGC | |
| | GTGCGGGGAAAGGACACCTCCGGATCCTACTACGCCATGGAC | |
| | TACTGGGGACAGGGCACCAGCGTGACTGTGTCCTCG | |
| SD- | CAGGTCCAGCTGGTAGAGTCCGGCGGCGGACTGGTCCAGCCT | 26 |
| 171467_VH | GGCGGAAGTCTGAAATTGTCTTGCGCTGCTAGCGGGTTCACT | |
| | TTCAATACAAATGCAATGAACTGGGTGCGCCAGGCAAGTGGC | |
| | AAAGGACTGGAGTGGGTCGGAAGAATCAGATCCAAGAGCAAT | |
| | AACTACGCCACTTACTATGCCGACTCCGTTAAGGATCGCTTC | |
| | ACGATATCCCGCGACGACAGTAAGAATACTCTCTACCTGCAG | |
| | ATGAACTCCTTAAAAACAGAAGACACTGCCGTATACTACTGT | |
| | GTAAGAGGTAAAGACACCTCAGGCTCCTATTATGCCATGGAC | |
| | TATTGGGGACAGGGTACAACCGTCACAGTGTCCAGC | |
| SD- | GAAGTGCAGCTGGTTGAATCCGGCGGGGGGCTGGTGCAGCCT | 27 |
| 962141_VH | GGAGGCTCCCTCAAGCTCTCTTGCGCTGCCTCCGGATTTACC | |
| | TTCAATACAAATGCGATGAACTGGGTGCGCCAGGCACCTGGG | |
| | AAAGGTTTGGAGTGGGTAGGCCGCATACGCAGCAAGAGCAAT | |
| | AACTATGCCACCTATTATGCAGATAGCGTAAAAGACAGATTC | |
| | ACTATCAGTCGGGACGACTCTAAATCCACCCTCTATCTGCAG | |
| | ATGAACTCACTGAAGACTGAGGACACCGCTGTCTATTACTGC | |
| | GTGCGGGGCAAGGATACGTCTGGTAGCTATTATGCTATGGAC | |
| | TATTGGGGCCAGGGGACACTGGTCACGGTTTCTTCT | |
| SD- | GAGGTGCAGCTGGTAGAGTCTGGGGGAGGGTTAGTCCAGCCG | 28 |
| 611534_VH | GGGGGTTCCCTGAAGCTCAGCTGTGCCGCCTCCGGGTTTACA | |
| | TTTTCCACCAACGCTATGAACTGGGTAAGACAGGCTCCCGGA | |
| | AAGGGACTCGAATGGGTGGGCCGCATTCGATCAAAGAGCAAC | |
| | AACTATGCCACGTACTACGCGGACTCAGTAAAAGATCGCTTC | |
| | ACTATATCCAGAGATGACTCTAAAAACACCTTGTACCTGCAG | |
| | ATGAACTCACTTAAAACTGAAGACACTGCTGTCTACTACTGC | |
| | GTAAGGGGCAAGGACACATCCGGCAGCTATTATGCCATGGAT | |
| | TACTGGGGTCAAGGTACCCTCGTTACAGTGAGCAGC | |
| SD- | CAGGTCCAGCTGGTAGAGTCCGGCGGCGGACTGGTCCAGCCT | 29 |
| 580600_VH | GGCGGAAGTCTGAAATTGTCTTGCGCTGCTAGCGGGTTCACT | |
| | TTCAATACAAATGCAATGAACTGGGTGCGCCAGGCAAGTGGC | |
| | AAAGGACTGGAGTGGGTCGGAAGAATCAGATCCAAGAGCAAT | |
| | AACTACGCCACTTACTATGCCGACTCCGTTAAGGATCGCTTC | |
| | ACGATATCCCGCGACGACAGTAAGAATACTCTCTACCTGCAG | |
| | ATGAACTCCTTAAAAACAGAAGACACTGCCGTATACTACTGT | |
| | GTAAGAGGTAAAGACACCTCAGGCTCCTATTATGCCATGGAC | |
| | TATTGGGGACAGGGTACAACCGTCACAGTGTCCAGC | |
| SD- | CAAGTGCAATTGGTCGAGTCCGGCGGGGGCCTGGTGCAACCT | 30 |
| 388950_VH | GGCGGGAGCCTGAAGCTCTCCTGCGCGGCAAGCGGGTTTACT | |
| | TTTAACACCAATGCCATGAATTGGGTTAGGCAAGCGTCCGGT | |
| | AAGGGATTGGAGTGGGTGGGGAGGATCCGATCAAAATCTAAT | |
| | AATTACGCCACCTATTACGCAGATTCTGTCAAAGATCGCTTT | |
| | ACTATCTCAAGAGATGACAGTCAGAGTATGCTCTATCTGCAA | |
| | ATGAACTCCCTGAAGACGGAAGACACAGCTGTTTACTATTGT | |
| | GTGCGGGGGAAGGACACTAGTGGTAGCTATTACGCCATGGAC | |
| | TACTGGGGTCAGGGAACCACAGTCACCGTGAGCTCT | |
| SD- | CAGGTCCAGCTGGTAGAGTCCGGCGGCGGACTGGTCCAGCCT | 31 |
| 117248_VH | GGCGGAAGTCTGAAATTGTCTTGCGCTGCTAGCGGGTTCACT | |
| | TTCAATACAAATGCAATGAACTGGGTGCGCCAGGCAAGTGGC | |
| | AAAGGACTGGAGTGGGTCGGAAGAATCAGATCCAAGAGCAAT | |
| | AACTACGCCACTTACTATGCCGACTCCGTTAAGGATCGCTTC | |
| | ACGATATCCCGCGACGACAGTAAGAATACTCTCTACCTGCAG | |
| | ATGAACTCCTTAAAAACAGAAGACACTGCCGTATACTACTGT | |
| | GTAAGAGGTAAAGACACCTCAGGCTCCTATTATGCCATGGAC | |
| | TATTGGGGACAGGGTACAACCGTCACAGTGTCCAGC | |
| SD- | GAGGTGCAGTTGGTCGAGAGTGGTGGGGGACTTGTACAGCCT | 32 |
| 166154_VH | GGGGGCTCACTGAAATTATCCTGTGCGGCGAGTGGATTTACT | |
| | TTTTCTACTAATGCTATGAATTGGGTTCGCCAGGCGAGCGGA | |
| | AAAGGCCTGGAATGGGTCGGGAGGATTCGCTCTAAATCTAAC | |
| | AACTATGCAACCTACTATGCCGACTCCGTTAAGGATCGCTTT | |
| | ACTATAAGCAGAGATGACAGCAAAAACACAGCGTATCTGCAA | |
| | ATGAACAGCCTCAAAACAGAGGACACAGCCGTTTACTACTGT | |
| | ACTAGAGGCAAAGACACTAGCGGTAGTTACTATGCCATGGAT | |
| | TACTGGGGTCAGGGGACGATGGTTACCGTGAGTTCC | |
| SD- | GAGGTGCAGTTGGTCGAGAGTGGTGGGGGACTTGTACAGCCT | 33 |
| 969683_VH | GGGGGCTCACTGAAATTATCCTGTGCGGCGAGTGGATTTACT | |
| | TTTTCTACTAATGCTATGAATTGGGTTCGCCAGGCGAGCGGA | |
| | AAAGGCCTGGAATGGGTCGGGAGGATTCGCTCTAAATCTAAC | |
| | AACTATGCAACCTACTATGCCGACTCCGTTAAGGATCGCTTT | |
| | ACTATAAGCAGAGATGACAGCAAAAACACAGCGTATCTGCAA | |
| | ATGAACAGCCTCAAAACAGAGGACACAGCCGTTTACTACTGT | |
| | ACTAGAGGCAAAGACACTAGCGGTAGTTACTATGCCATGGAT | |
| | TACTGGGGTCAGGGGACGATGGTTACCGTGAGTTCC | |

TABLE 3B
Anti-CCR8 humanized antibody nucleic acid
sequences - light chains.

| | | SEQ ID |
|---|---|---|
| Seq ID | VL Seq | NO: |
| SD-356253_VL | GACATCGTGATGACTCAGGCCGCCCCTTCCGTGCCCGTC | 34 |
| | ACCCCGGGAGAATCCGTGTCAATCTCGTGTCGGTCCTCC | |
| | AAGTCGCTGCTTCATTCCAACGGGAACACCTATCTGTAC | |
| | TGGTTCTTGCAAAGACCCGGACAGAGCCCGCAGCTGCTG | |
| | ATCTACCGCATGTCTAACCTCGCTAGCGGAGTGCCAGAT | |
| | CGGTTTTCGGGATCCGGCAGCGGCACCGCGTTCACTCTG | |
| | CGCATTTCAAGGGTCGAAGCCGAGGACGTGGGCGTGTAC | |
| | TACTGCATGCAGCACCTGGAGTACCCTTTCACCTTCGGG | |
| | TCCGGTACCAAGCTCGAAATTCGG | |
| SD-171467_VL | GATATTGTGATGACTCAGTCGCCGCTGTCATTGCCCGTG | 35 |
| | ACCCCTGGAGAACCCGCGTCGATTTCCTGTCGGTCCTCC | |
| | AAGTCCCTCCTGCACTCCAACGGGAACACCTACCTCTAC | |
| | TGGTTTCTGCAAAAGCCGGGACAGAGCCCACAGCTGCTG | |
| | ATCTATCGGATGAGCAACCTTGCCTCGGGAGTGCCTGAC | |
| | CGCTTCTCCGGAAGCGGCTCAGGGACCGACTTCACCCTG | |
| | AAAATCTCCAGAGTGGAGGCCGAAGATGTCGGCGTGTAC | |
| | TACTGCATGCAGCATCTGGAGTACCCGTTCACTTTCGGC | |
| | GGTGGTACCAAGGTCGAAATCAAG | |
| SD-962141_VL | GATATTGTGATGACTCAGTCGCCGCTGTCATTGCCCGTG | 36 |
| | ACCCCTGGAGAACCCGCGTCGATTTCCTGTCGGTCCTCC | |
| | AAGTCCCTCCTGCACTCCAACGGGAACACCTACCTCTAC | |
| | TGGTTTCTGCAAAAGCCGGGACAGAGCCCACAGCTGCTG | |
| | ATCTATCGGATGAGCAACCTTGCCTCGGGAGTGCCTGAC | |
| | CGCTTCTCCGGAAGCGGCTCAGGGACCGACTTCACCCTG | |
| | AAAATCTCCAGAGTGGAGGCCGAAGATGTCGGCGTGTAC | |
| | TACTGCATGCAGCATCTGGAGTACCCGTTCACTTTCGGC | |
| | GGTGGTACCAAGGTCGAAATCAAG | |
| SD-611534_VL | GATATTGTGATGACTCAGTCGCCGCTGTCATTGCCCGTG | 37 |
| | ACCCCTGGAGAACCCGCGTCGATTTCCTGTCGGTCCTCC | |
| | AAGTCCCTCCTGCACTCCAACGGGAACACCTACCTCTAC | |
| | TGGTTTCTGCAAAAGCCGGGACAGAGCCCACAGCTGCTG | |
| | ATCTATCGGATGAGCAACCTTGCCTCGGGAGTGCCTGAC | |
| | CGCTTCTCCGGAAGCGGCTCAGGGACCGACTTCACCCTG | |
| | AAAATCTCCAGAGTGGAGGCCGAAGATGTCGGCGTGTAC | |
| | TACTGCATGCAGCATCTGGAGTACCCGTTCACTTTCGGC | |
| | GGTGGTACCAAGGTCGAAATCAAG | |
| SD-580600_VL | GACATTGTTATGACCCAGTCTGCCTTATCTCTGCCCGTA | 38 |
| | ACCCCTGGGGAGCCCGCGAGCATAAGTTGCAGGTCCTCA | |
| | AAAAGCCTTTTGCATAGTAATGGCAATACATACTTGTAT | |
| | TGGTTTCTGCAGAAGCCTGGACAGAGCCCTCAGCTGCTG | |
| | ATTTATAGAATGAGCAACCTCGCTTCTGGAGTGCCCGAC | |
| | CGCTTTAGCGGCTCTGGATCAGGGACTGATTTTACACTG | |
| | AA39GATAAGCAGAGTGGAAGCTGAGGATGTTGGCGTGT | |
| | ATTACTGTATGCAGCATCTCGAGTATCCATTTACATTTG | |
| | GTGGGGGCACTAAGGTGGAAATCAAG | |
| SD-388950_VL | GACATTGTTATGACCCAGTCTGCCTTATCTCTGCCCGTA | 39 |
| | ACCCCTGGGGAGCCCGCGAGCATAAGTTGCAGGTCCTCA | |
| | AAAAGCCTTTTGCATAGTAATGGCAATACATACTTGTAT | |
| | TGGTTTCTGCAGAAGCCTGGACAGAGCCCTCAGCTGCTG | |
| | ATTTATAGAATGAGCAACCTCGCTTCTGGAGTGCCCGAC | |
| | CGCTTTAGCGGCTCTGGATCAGGGACTGATTTTACACTG | |
| | AAGATAAGCAGAGTGGAAGCTGAGGATGTTGGCGTGTAT | |
| | TACTGTATGCAGCATCTCGAGTATCCATTTACATTTGGT | |
| | GGGGGCACTAAGGTGGAAATCAAG | |
| SD-117248_VL | GATATCGTCATGACTCAGAGCCCCCTGTCCCTGCCGGTG | 40 |
| | ACCCCCGGCGAGCCAGCGTCCATTTCCTGTCGCAGCTCT | |
| | AAATCACTGCTGCATTCTAATGGCAATACCTACTTGTAC | |
| | TGGTTCCTTCAGAAACCAGGCCAGTCACCACAGCTGCTG | |
| | ATATATCGTATGTCTAATTTGGCGAGCGGCGTGCCTGAC | |
| | CGATTCTCAGGCTCTGGCAGCGGCACTGCTTTTACCCTG | |
| | AAGATTAGTCGGGTCGAGGCCGAAGATGTGGGAGTCTAC | |
| | TACTGTATGCAGCATCTGGAATACCCTTTTACTTTTGGG | |
| | GGCGGCACTAAGGTAGAGATTAAA | |
| SD-166154_VL | GATATCGTCATGACTCAGAGCCCCCTGTCCCTGCCGGTG | 41 |
| | ACCCCCGGCGAGCCAGCGTCCATTTCCTGTCGCAGCTCT | |
| | AAATCACTGCTGCATTCTAATGGCAATACCTACTTGTAC | |
| | TGGTTCCTTCAGAAACCAGGCCAGTCACCACAGCTGCTG | |
| | ATATATCGTATGTCTAATTTGGCGAGCGGCGTGCCTGAC | |
| | CGATTCTCAGGCTCTGGCAGCGGCACTGCTTTTACCCTG | |
| | AAGATTAGTCGGGTCGAGGCCGAAGATGTGGGAGTCTAC | |
| | TACTGTATGCAGCATCTGGAATACCCTTTTACTTTTGGG | |
| | GGCGGCACTAAGGTAGAGATTAAA | |
| SD-969683_VL | GACATAGTGATGACCCAGAGTCCATTATCCCTGCCAGTC | 42 |
| | ACGCCTGGGGAGTCAGTGAGCATTTCCTGCCGATCCAGT | |
| | AAAAGCCTCCTTCATTCCAATGGTAATACATACCTGTAC | |
| | TGGTTCCTCCAGAAACCCGGCCAATCTCCTCAGTTACTT | |
| | ATTTACAGAATGTCCAACCTCGCATCTGGGGTGCCCGAC | |
| | AGGTTTTCCGGAAGTGGGTCAGGCACTGACTTCACCCTG | |
| | AGGATCAGTAGGGTGGAGGCCGAGGATGTGGGCGTGTAC | |
| | TACTGCATGCAGCATCTCGAGTACCCTTTCACCTTCGGG | |
| | GGAGGAACAAAAGTAGAGATAAAA | |

TABLE 4
Additional CDR sequences

| | Antibody | | |
|---|---|---|---|
| | CDR1 | CDR2 | CDR3 |
| 1E6H3K - HC | GFTFNTNA | IRSKSNNYAT | VRGKDTSGS |
| | | | YYAMDY |
| SEQ ID NO: | 43 | 44 | 45 |
| 1E6H3K - LC | KSLLHSNGNTY | RMS | MQHLEYPFT |
| SEQ ID NO: | 46 | 47 | 48 |
| 7B_600_030 - | GFIFETYA | IRSKSNNYAT | VRGLLRYRF |
| HC | | | FDV |
| SEQ ID NO: | 49 | 50 | 51 |
| 7B_600_030 - | KSLLHSNGNTY | KVE | MQHLEYPFT |
| LC | | | |
| SEQ ID NO: | 52 | 53 | 54 |
| 7B_000_032 | GFTFNTYA | IRSKSNNYAT | VRGLLRYRF |
| | | | FDV |
| SEQ ID NO: | 55 | 56 | 57 |
| 7B_000_032 | KSLLHSNGNTY | KVE | QQHTNWPFT |
| SEQ ID NO: | 58 | 59 | 60 |
| 7B_600_022 | GFIFETYA | IRSKSNNYAT | VRGLLRYRF |
| | | | FDV |
| SEQ ID NO: | 61 | 62 | 63 |
| 7B_600_022 | KSLLHSNGNTY | KAN | QQHTNWPFT |
| SEQ ID NO: | 64 | 65 | 66 |
| 7B_600_032 - | GFIFETYA | IRSKSNNYAT | VRGLLRYRF |
| HC | | | FDV |
| SEQ ID NO: | 67 | 68 | 69 |
| 7B 600_032 - | KSLLHSNGNTY | KVE | QQHTNWPFT |
| LC | | | |
| SEQ ID NO: | 70 | 71 | 72 |
| 7B_600_012 - | GFIFETYA | IRSKSNNYAT | VRGLLRYRF |
| HC | | | FDV |
| SEQ ID NO: | 73 | 74 | 75 |
| 7B_600_012 - | KSLLHSNGNTY | GLT | QQHTNWPFT |
| LC | | | |
| SEQ ID NO: | 76 | 77 | 78 |
| 7B_600_020 - | GFIFETYA | IRSKSNNYAT | VRGLLRYRF |
| HC | | | FDV |
| SEQ ID NO: | 79 | 80 | 81 |
| 7B_600_020 - | KSLLHSNGNTY | KAN | MQHLEYPFT |
| LC | | | |
| SEQ ID NO: | 82 | 83 | 84 |
| K17_050_050 - | GYTFTSYN | VHPGSGST | ARKGGTPFA |
| HC | | | Y |
| SEQ ID NO: | 85 | 86 | 87 |
| K17_050_050 - | QSLVHSNAATY | QVK | SQSTHVPYT |
| LC | | | |
| SEQ ID NO: | 88 | 89 | 90 |

In some embodiments, provided herein is an anti-CCR8 antibody, wherein the antibody comprises the amino acid sequences of the following a heavy chain variable domain (VH) complementarity determining region (CDR) 1 comprising the amino acid sequence of any one of the following SEQ ID NOS: 1, 49, 67, 73, 79, or 85; and a VH CDR2 comprising the amino acid sequence of any one of the following SEQ ID NOS: 2, 50, 68, 74, 80, or 86; and a VH CDR3 comprising the amino acid sequence of any one of the following SEQ ID NOS: 3, 51, 69, 75, 81, or 87; and a light chain variable domain (VL) CDR1 comprising the amino acid sequence of any one of the following SEQ ID NOS: 4, 52, 55, 58, 61, 64, 70, 76, 82, or 88; and a VL CDR2 comprising the amino acid sequence of any one of the following SEQ ID NOS: 5, 53, 56, 59, 62, 65, 71, 77, 83, or 89; and a VL CDR3 comprising the amino acid sequence of any one of the following SEQ ID NOS: 6, 54, 57, 60, 63, 66, 72, 78, 84, or 90.

Exemplary Tandem scFv-Fc anti-CCR8 Antibodies. In some embodiments, the disclosure provides for tandem scFv antibodies, with multiple CCR8 binding sites. Tandem scFv-Fc antibodies of the disclosure are composed two or more scFv binding sites in tandem on each antibody arm, optionally linked by a linker, optionally a flexible linker. In some embodiments, a tandem scFV antibody has a total of four or more scFv binding sites in a single scFv-Fc formatted antibody.

In some embodiments, the scFv1 of each antibody arm comprises a first heavy chain variable domain (VH1) and a first light chain variable domain (VL1); and the scFv2 of each antibody arm comprises a first heavy chain variable domain (VH2) and a first light chain variable domain (VL2). The VH1 and the VL1 of each scFV1 may be connected by a linker, e.g., a flexible linker. The VH2 and the VL2 of each scFV2 may be connected by a linker, e.g., a flexible linker. The scFvs on each antibody arm may be connected by a linker, e.g., a flexible linker.

Therapeutic Uses of Anti-CCR8 Antibodies.

In some embodiments, the anti-CCR8 antibodies provided herein are useful for the treatment of a disease or condition involving an immune response.

Administration of Therapeutic anti-CCR8 Antibodies. The in vivo administration of the therapeutic anti-CCR8 antibodies described herein may be carried out intravenously, intramuscularly, subcutaneously, topically, orally, transdermally, intraperitoneally, intraorbitally, intrathecally, intraventricularly, intranasally, transmucosally, through implantation, or through inhalation. Intravenous administration may be carried out via injection or infusion. In some embodiments, the anti-CCR8 antibodies of the disclosure are administered intravenously. In some embodiments, the anti-CCR8 antibodies of the disclosure are administered subcutaneously. Administration of the therapeutic anti-CCR8 antibodies may be performed with any suitable excipients, carriers, or other agents to provide suitable or improved tolerance, transfer, delivery, and the like.

### Examples

### Example 1: Anti-CCR8 Discovery Based on Engineered MEM-Nanoparticle Immunizations

MEMs were designed to embody the structure and dynamics of extracellular loop 1 (DQWVFGT)(SEQ ID NO:91) and extracellular loop 2 (VASEDGVLQC) (SEQ ID NO:92) of CCR8. Keeping the epitope residues for extracellular loops 1 and 2 fixed, a protein design AI algorithm was used to build a protein scaffold to present the epitope structure. The scaffold sequence was optimized to satisfy three criteria: [1] structural match to the predicted epitope structure in CCR8, [2] stability of the MEM molecule, and [3] water solubility. The length of the protein scaffold was optimized to be as small as possible without sacrificing on these three criteria. The amino acid sequence of the final CCR8 MEM design is SPEIKKLIEQIKSDQWVFGTEACNEIQTLLKEESGPAKIEVASEDGVLQCRIVF (SEQ ID NO:93). The structure of this MEM design was validated by nuclear magnetic resonance (NMR) to be within 3.2 Å RMSD of the predicted structure. When only considering the epitope residues, the RMSD between the experimental and predicted structures is 1.6 Å.

The CCR8 MEM was expressed and purified as a nanoparticle fusion protein with ferritin from Helicobacter pylori. The MEM was joined at the N-terminus of each ferritin subunit using linker sequence GGGGS (SEQ ID NO:94). These nanoparticles were expressed and purified in E. coli by nickel-NTA affinity chromatography.

BALB/c mice were immunized in two separate cohorts. In one cohort, immunizations alternated between MEM immunizations one week and CCR8 virus-like particles (VLP) (Acro Biosystems, Cat ##CC8-H52P4) every other week. In a separate cohort, mice were immunized with CCR8 expressing HEK293 and/or CHO cells one week, and were immunized with CCR8 VLPs every other week. MEM immunizations were 15-25 ug per injection and CCR8 VLPs were 6 ug per injection.

### Example 2: Top Monoclonal Hybridoma Produced Antibody Demonstrates Strong Anti-CCR8 Binding, does not Bind CCR4, and Competes with GS-1811 Benchmark

Hybridoma produced antibody was incubated with cells transiently expressing either CCR8 or CCR4 for 1 hour, washed once with DPBS+1% FBS, then labeled with anti-mouse IgG secondary antibody for 1 hour. Cells were then washed again and measured by flow cytometry. For cross blocking studies, the hybridoma produced antibody was co-incubated with GS-1811 as a human IgG1 for 1 hour, washed once with DPBS+1% FBS, then labeled with anti-mouse IgG secondary antibody for 1 hour. Cells were then washed 1× and measured by flow cytometry. All measurements were performed on ice and with chilled buffers.

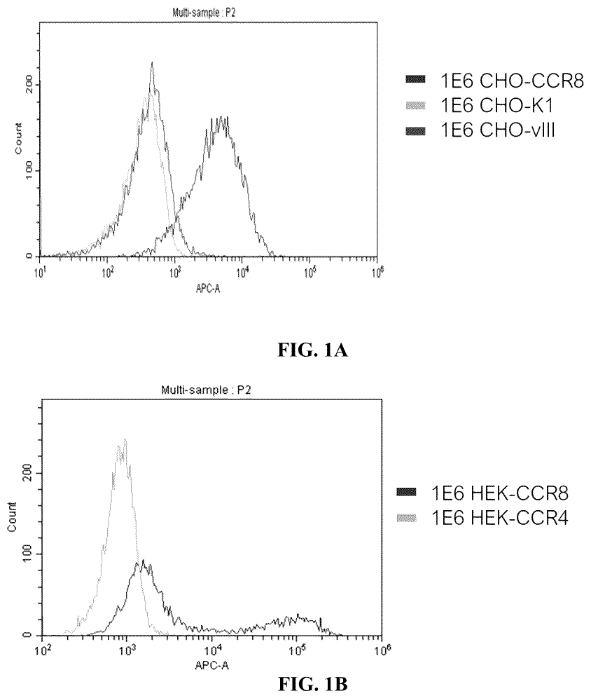

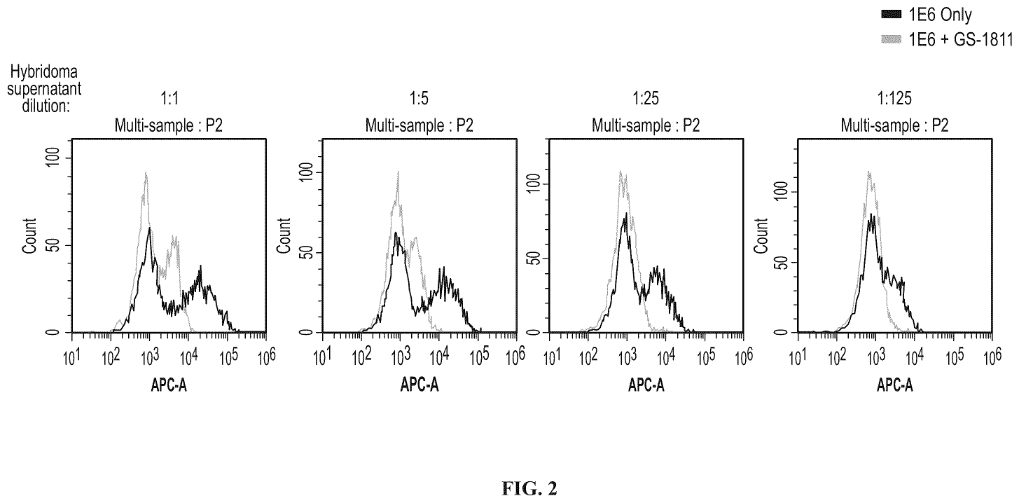

FIGS. 1A and 1B show that anti-CCR8 hybridoma hit clone 1E6 specifically binds CCR8 expressing cells and does not bind CCR4 expressing cells. FIG. 2 shows that the 1E6 hybridoma clone competes with GS-1811 benchmark in binding to CCR8 expressing cells.

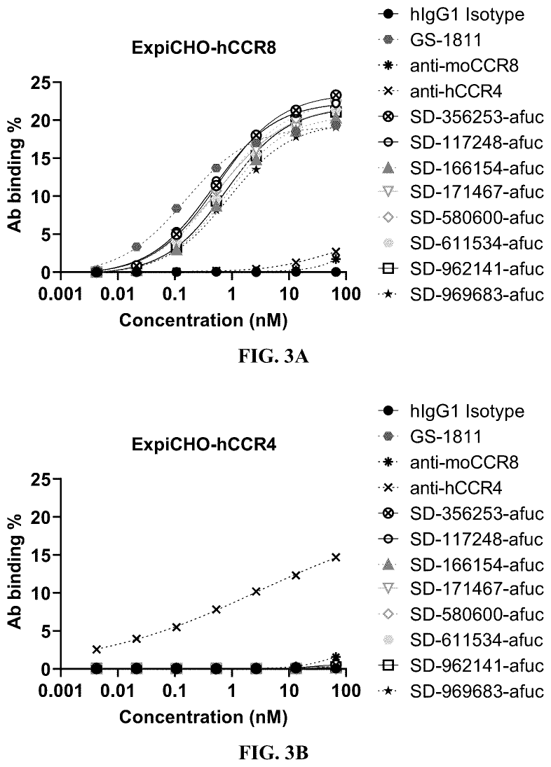

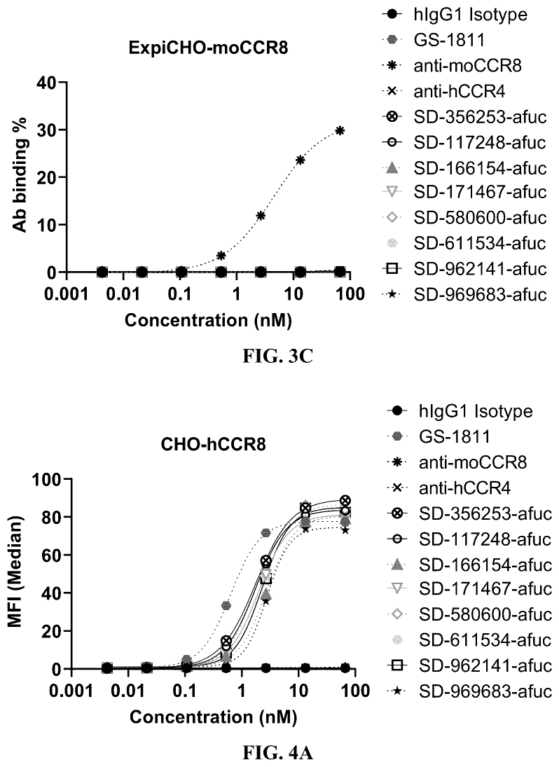

FIGS. 3A to 3C show that anti-CCR8 antibodies derived from chimera or humanization of hybridoma clone 1E6 binds specifically to human CCR8 and not human CCR4 or mouse CCR8. FACS analysis showing the binding of anti-CCR8 antibodies or control antibodies (hIgG1 isotype, anti-mouse CCR8, and anti-human CCR4) against ExpiCHO cells expressing human CCR8, human CCR4, or mouse CCR8. Values plotted are the percentage of antibody bound to the target cell.

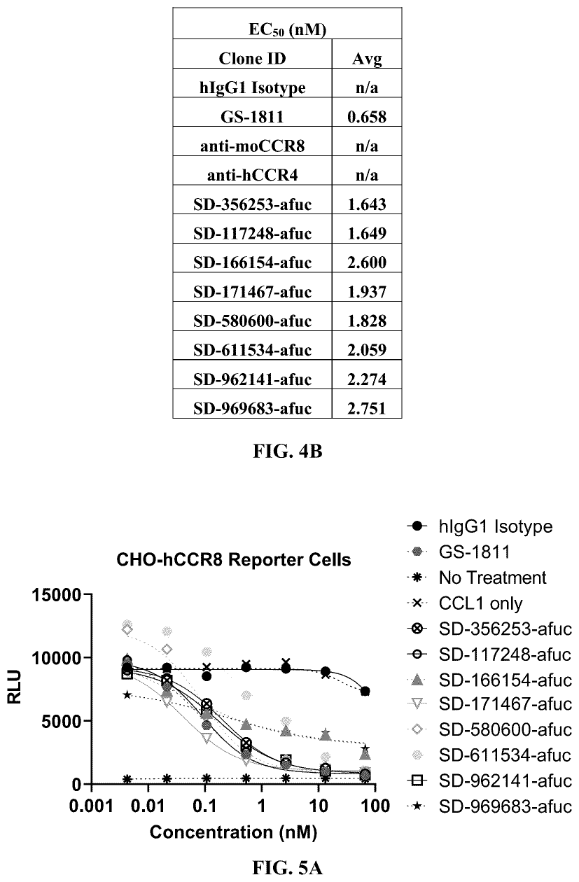

FIGS. 4A and 4B show binding potency of anti-human CCR8 antibodies to CHO-K1 cell line overexpressing human CCR8. FACS analysis showing the binding of anti-CCR8 antibodies or control antibodies (hIgG1 isotype, anti-mouse CCR8, and anti-human CCR4) against CHO-K1 cells overexpressing human CCR8. Values plotted are Median Fluorescent Intensity. EC₅₀ values are averages of n=2 experiments. n/a: no activity.

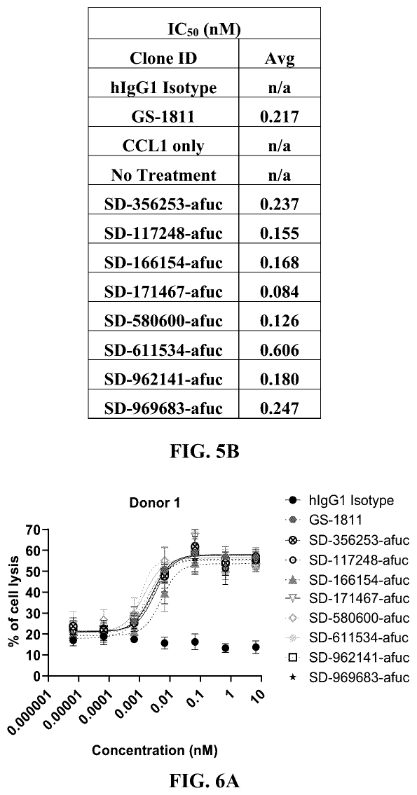

FIGS. 5A and 5B show the antagonist activity of anti-human CCR8 antibodies in CHO-K1 reporter cell line overexpressing human CCR8. Luminescence readout showing the inhibition of human CCR8 activation with the agonist CCL1 by pre-treating the reporter cells with anti-CCR8 antibodies. Values plotted are Relative Luminescence Units. EC₅₀ values are averages of n=2 experiments. n/a: no activity.

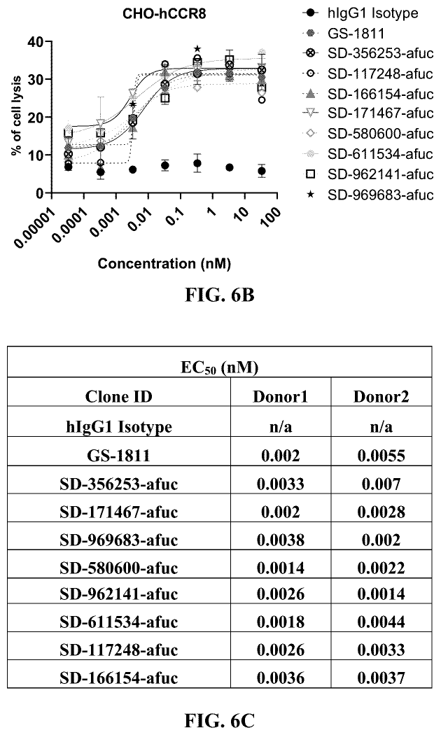

FIGS. 6A to 6C show the antibody-dependent cell cytotoxicity (ADCC) activity of the anti-CCR8 antibodies using human peripheral blood mononuclear cells (PBMCs). FACS analysis showing the ADCC potency of anti-CCR8 antibodies in a dose-dependent manner from two different PBMC donors. Values plotted are the percentage of dead target cell (% of cell lysis) to the total target cell. EC₅₀ values are averages of n=1-2 experiments. n/a: no activity

FIG. 7 is a graph that shows the thermal stability of the anti-CCR8 antibodies. Melting temperature (T_{M}) of CCR8 binding antibodies was determined using a Prometheus Panta instrument by monitoring the change in fluorescent intensity of antibody loaded into a capillary as temperature was slowly increased.

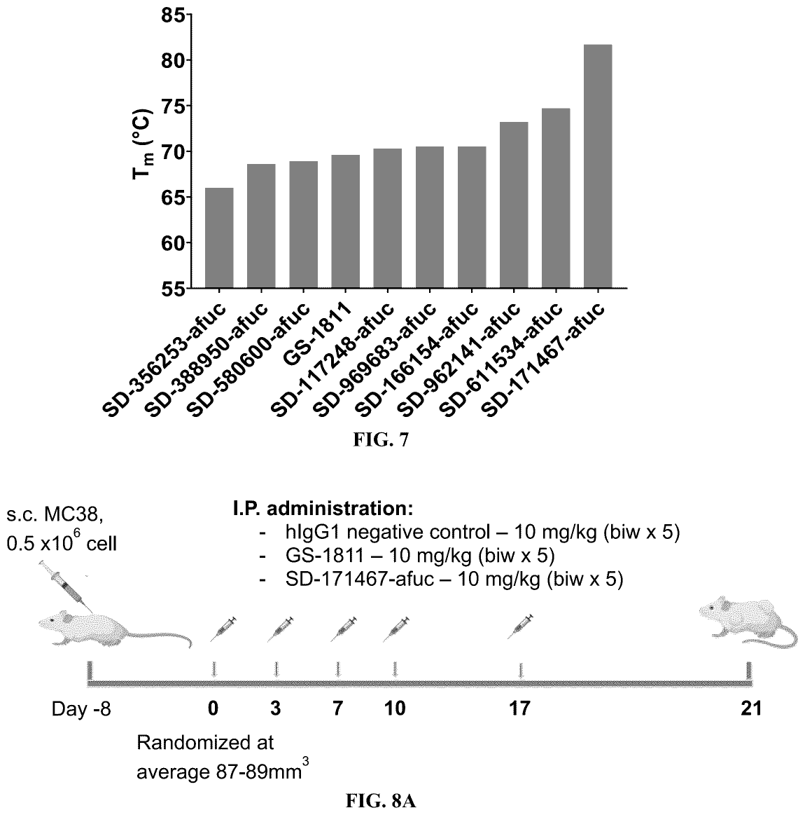

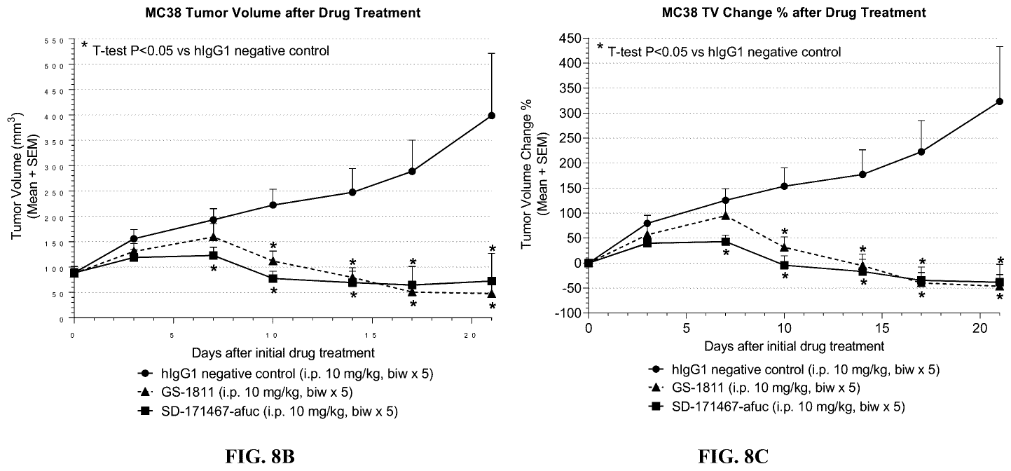

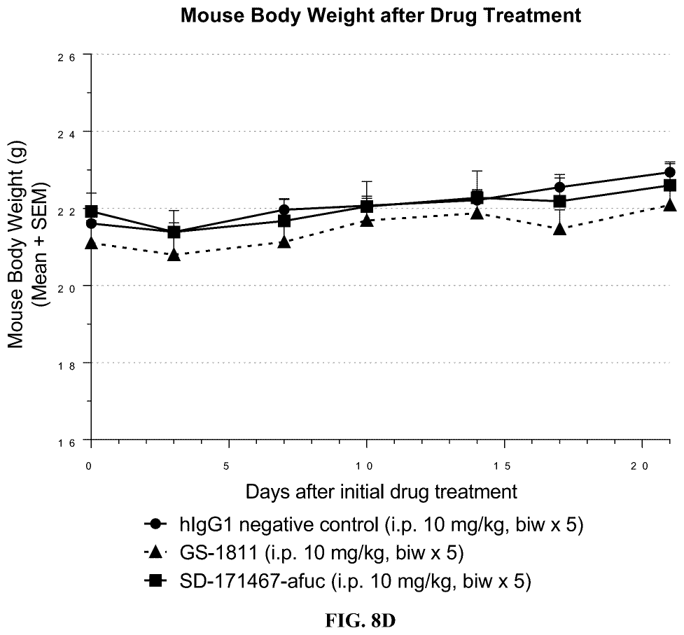

FIGS. 8A to 8D shows the study design and results for the in vivo efficacy study using anti-CCR8 antibody SD-171467-afuc as a monotherapy. FIGS. 8B and 8C shows tumor volume and percent tumor volume change after drug treatment. Tumor measurements and drug administrations were on days 0, 3, 7, 10 and 17. FIG. 8D shows the changes in mouse body weight from drug administrations on days 0, 3, 7, 10 and 17. For all regimens, no significant mouse body weight loss was observed after drug treatment.

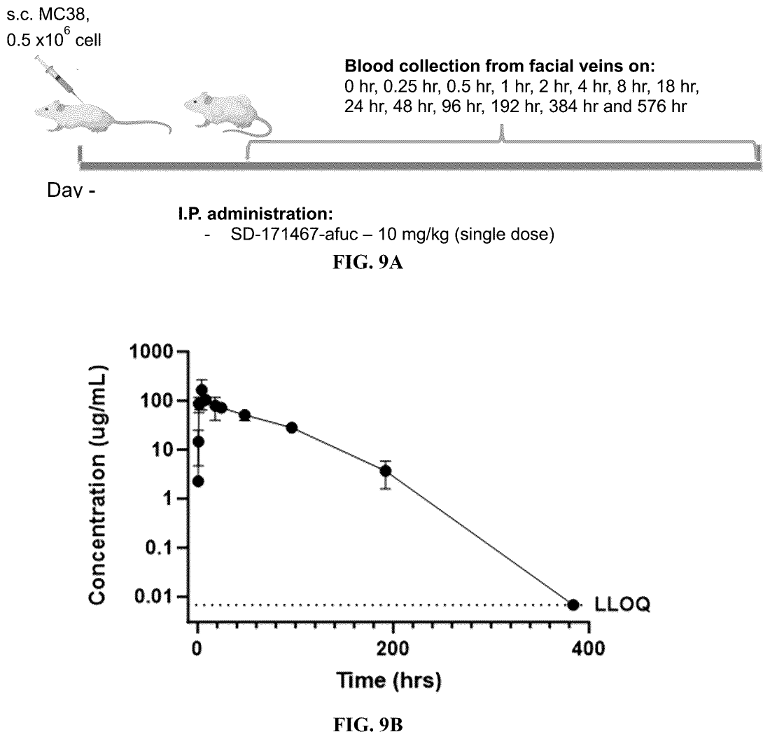

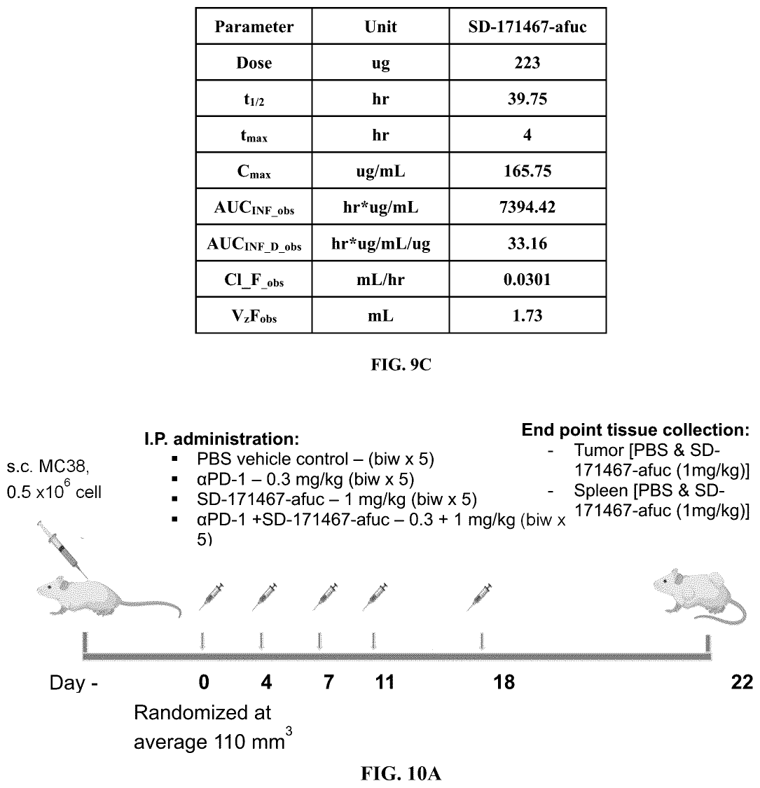

FIGS. 9A to 9C show the results of the pharmacokinetic studies. FIG. 9A shows the pharmacokinetic study design for SD-171467-afuc. FIG. 9B shows the pharmacokinetic profile of SD-171467-afuc. Serum was collected after a single 10 mg/kg dose at the indicated time points. Human IgG levels was quantified using the MSD Human IgG kit. FIG. 9C summarizes the pharmacokinetic parameters calculated by Phoenix WinNonLin.

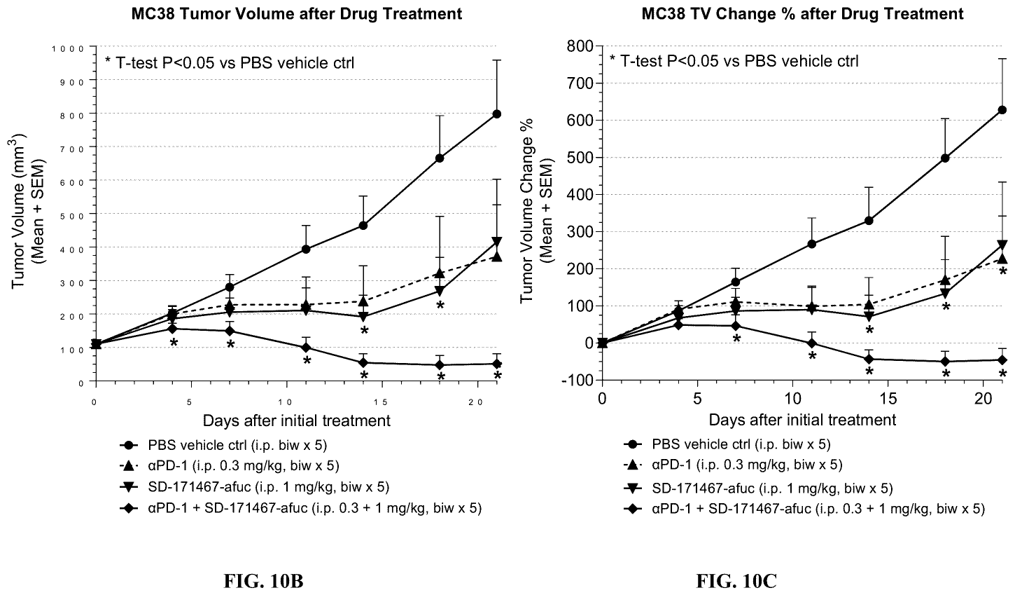

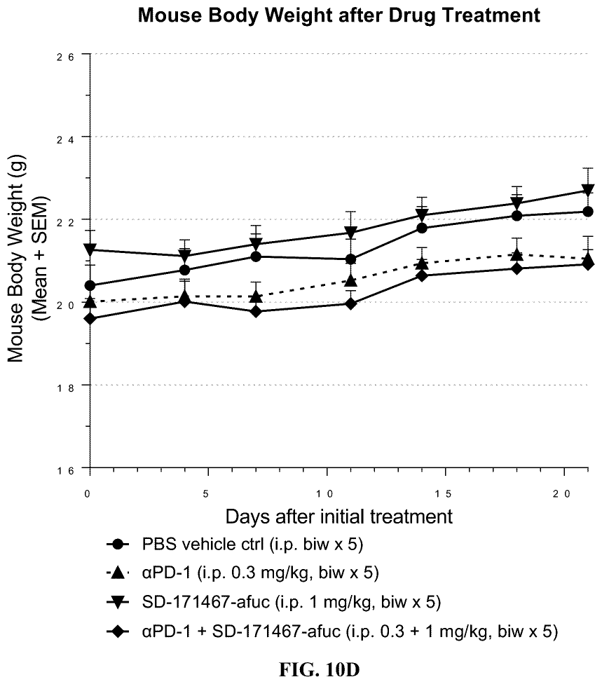

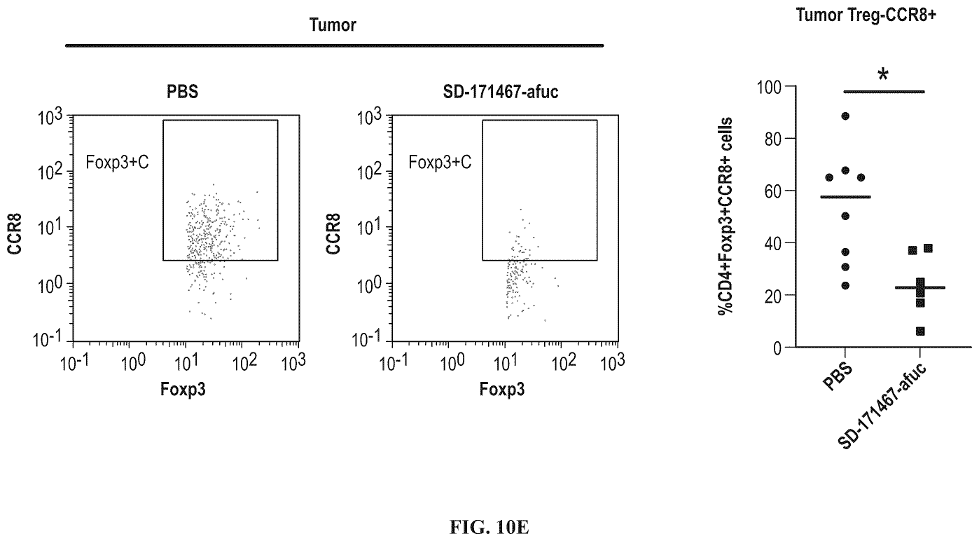

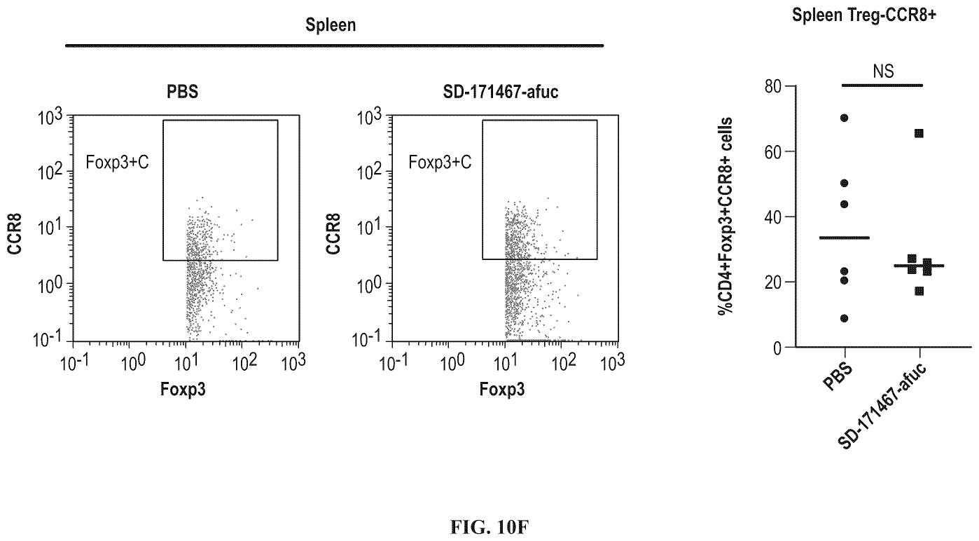

FIGS. 10A to 10F show the study design and results for the in vivo efficacy study using SD-171467-afuc and anti-PD1 antibody as combination treatment and the mechanism of action study for CCR8+ Treg depletion. FIG. 10A shows the in vivo study design for SD-171467-afuc combination treatment with anti-PD1 antibody and CCR8+ Treg depletion. FIGS. 10B and 10C show tumor volume and percent tumor volume change after drug treatment. Tumor measurements and drug administrations were on days 0, 4, 7, 11, and 18. FIG. 10D shows the changes in mouse body weights from drug administrations on days 0, 4, 7, 11, and 18. For all regimens, no significant mouse body weight loss was observed after drug treatment. FIGS. 10E and 10F show that treatment with SD-171467-afuc significantly decreased Treg-CCR8+ in tumor but not in spleen. Tumor (FIG. 10E) and spleen (FIG. 10F) samples from PBS and SD-171467-afuc (1 mg/kg) treatment groups were analyzed for Treg-CCR8+ depletion using flow cytometry. Graphs show the percentages of Treg-CCR8+ (CD4+Foxp3+CCR8+) out of live CD45+CD3+CD4+Foxp3+ cells in the tumor and spleen. Student t-Test, two tailed, was used to compare the two treatment groups. The p value is shown in the graphs (*p<0.05; ^{NS}p>0.05).

Hybridomas were created from the immunized mouse B-cells using standard electro cell fusion methods. Spleens were isolated from immunized mice and ground in 15-20 mL of DMEM (no additions) on ice. Splenocytes and Ag8.653 mouse myeloma cells were mixed in a 1:1 ratio in DMEM on ice. Cells were resuspended in a volume of ECF buffer such that the density is 2×10⁶ cells/mL before performing electrofusion. Fusion cycle parameters (Nepagene/ECFG21) were used to perform the electrofusion. Cells were plated in 96-well plates containing selection medium and verify fusion efficiency 7 days after the fusion.

Antibody expression and expression. Antibody expression plasmids were transiently introduced into an animal cell line using the ExpiFectamine CHO Transfection Kit (Thermo Fisher, Cat #A29129) to yield transfectants that produced anti-CCR8 chimeric or humanized antibody. For a host cell line, ExpiCHO-S (Thermo Fisher, Cat #A29127) or a suspension CHO cell line with the α1,6 fucosyltransferase (FUT8) gene knocked out were used (referred to as “WT CHO” and “FUT8 CHO” in further references). After 6-12 days of growth post introduction of DNA, cell suspensions of WT CHO or FUT8 CHO were harvested via centrifugation for 20 min at 4,000×g, and then filtered using 0.2 μm Disposable PES Filter units (Fisher Scientific, Cat #FB12566504). Anti-CCR8 antibody was recovered from filtrate using Protein A purification (HiTrap MabSelect SuRe; Cytiva, Cat #GE11-0034-93). WT CHO was used to express antibodies with standard glycosylation, and FUT8 CHO used to express afucosylated antibodies with enhanced effector function (indicated by “-afuc”).

Antibody humanization. Humanization was accomplished via multiple approaches. In some instances, a publicly available tool (DOI: 10.1080/19420862.2021.2020203) was used to graft the CDRs directly onto a human germline. To potentially improve expression and performance, single amino acids were reverted to the parental mouse sequence. In other cases, two or more amino acids were reverted to the parental mouse sequence. In all cases, the CDRs were left unchanged. Humanized variants were then tested and potency compared to the parental chimera.

Thermal stability. Antibodies were prepared at a concentration of 1 mg/mL in PBS (pH 7.4). Ten microliters of each sample were loaded into an appropriate capillary (Nanotemper, Cat #PR-0002) via pipette. Capillaries were loaded into a Nanotemper Prometheus Panta instrument, and protocol defined. Thermal unfolding was performed from 25-95° C. at a rate of 1° C./min. Fluorescence was monitored and Tₘ calculated by the instrument's software.

Cell Binding Assay. PathHunter CHO-K1 β-Arrestin cells (DiscoverX, Cat #93-0196C2) were cultured in AssayComplete Cell Culture Kit-107 (DiscoverX, Cat #92-3107G) supplemented with G418 (Gibco, Cat #10131-035) and Hygromycin B (Invitrogen, Cat #10687010) according to manufacturer's instructions. ExpiCHO-S cells (Thermo Fisher, Cat #A29127) were cultured in ExpiCHO expression medium (Gibco, Cat #A29100-01).

To generate human CCR8, human CCR4, or mouse CCR8 expressing cells, ExpiCHO cells were diluted to 6×10⁶ cells/mL and transfected with the corresponding expression plasmids using ExpiFectamine CHO transfection kit (Gibco, Cat #A29129) according to manufacturer's instructions. Transfected cells were used for cell binding assay within 5 days post-transfection. To detect human CCR4 or mouse CCR8 in the transfected cells, anti-hCCR4 (BioLegend, Cat #359402) or anti-moCCR8 (BioLegend, Cat #150302) were used for staining, respectively.

For the cell binding assay, PBS (Corning, Cat #21-040-CV) supplemented with 2% FBS (MilliporeSigma, Cat #F4135) and 2 mM EDTA (Quality Biological, Cat #351-027-721) was used as the assay buffer. Cells were counted then resuspended in the assay buffer, then seeded into 96-well plates (VWR, Cat #89089-826) at 1×10⁵ cells/well. The plates were centrifuged and kept on ice for the remainder of the assay after the supernatant was removed. Following supernatant removal, the indicated antibodies were diluted in the assay buffer and added to the cells at increasing concentrations (0.004-66.66 nM) for 20 minutes on ice. After incubation, the plates were centrifuged and the supernatant was removed, then the cells were washed once with the assay buffer followed by centrifugation and wash removal. After wash, rat-anti-human IgG Fc Alexa Fluor 647 (BioLegend, Cat #410714), rat-anti-mouse IgG Alexa Fluor 647 (BioLegend, Cat #406618), or goat-anti-rat IgG Alexa Fluor 647 secondary (BioLegend, Cat #405416) was added to the cells at 1:200 dilution in the assay buffer, for 20 minutes on ice. After incubation, the plates were centrifuged and the supernatant was removed, then the cells were washed once with the assay buffer followed by centrifugation and wash removal. After wash, DAPI (BioLegend, Cat #422801) was added to the cells at 1:5000 dilution in the assay buffer. Cell binding was analyzed on a Miltenyi MACSQuant 16 flow cytometer. Flow cytometry data were analyzed with the FlowJo flow cytometry analysis software. For making graphs and calculation of the EC₅₀ values GraphPad Prism 9.3.0 was used.

CCR8 Antagonist Assay. PathHunter CHO-K1 β-Arrestin cells (DiscoverX, Cat #93-0196C2) were cultured in AssayComplete Cell Culture Kit-107 (DiscoverX, Cat #92-3107G) supplemented with G418 (Gibco, Cat #10131-035) and Hygromycin B (Invitrogen, Cat #10687010) according to manufacturer's instructions.

For the CCR8 antagonist assay, PathHunter CHO-K1 β-Arrestin cells were detached with AssayComplete Cell Detachment Reagent (DiscoverX, Cat #92-0009), then counted and seeded at 2.5×10⁴ cells/well using AssayComplete Cell Plating 2 Reagent in 96-well white opaque microplates (Corning, Cat #3917) overnight at 37° C. and 5% CO₂. Next, the indicated antibodies were diluted in AssayComplete Cell Plating 2 Reagent at 10× of the final concentration and added to the cells at increasing concentration (10× final concentration range: 0.04-666.6 nM). The cells were incubated with the antibodies for 30 minutes at 37° C. and 5% CO₂, followed by the addition of CCL1 (PeproTech, Cat #300-37) agonist to the cells at 10× of the determined EC 80 value for the agonist. The cells were incubated with the antibodies and agonist (now at 1× final concentration for both) for 90 minutes at 37° C. and 5% CO₂. After agonist incubation, detection substrate was generated and added to the cells using the PathHunter Detection Kit (DiscoverX, Cat #93-0001), according to manufacturer's instruction. Cells were incubated at room temperature for 1 hour with the detection substrate, then analyzed on the Agilent Cytation 5 Cell Imaging/Multimode Reader with the following settings: Read speed: 1 second/well, read height: 1 mm, Gain: 135. The IC₅₀ value was calculated using Graphpad Prism 9.3.0.

ADCC/PBMC. PathHunter CHO-K1 β-Arrestin cells (DiscoverX, Cat #93-0196C2) were cultured in AssayComplete Cell Culture Kit-107 (DiscoverX, Cat #92-3107G) supplemented with G418 (Gibco, Cat #10131-035) and Hygromycin B (Invitrogen, Cat #10687010) according to manufacturer's instructions.

For ADCC/PBMC assay, PathHunter CHO-K1 β-Arrestin cells were counted to assess the cell number and viability. The cells were stained with cell-trace CFSE (Thermo Fisher, Cat #C345554A). Cells were centrifuged and resuspended in growth media at 1×10⁵ cells/mL and seeded at 1×10⁴ cells per well onto 96-well plates (VWR, Cat #29442-056) and incubated in cell culture incubator overnight at 37° C. and 5% CO 2. After incubation, indicated antibodies in assay media (RPMI 1640, with 10% FBS, 1% Penicillin/Streptomycin, and 5 ng/mL IL2) were added to the cells at increasing concentrations (0.00000667-6.666667 nM or 0.0000333-33.33333 nM) for 20 minutes at 37° C., 5% CO 2. Afterwards, 2×10⁵ peripheral blood mononuclear cells (PBMC) (Stemcell, Cat #70025.1) were added into each well of the 96-well plate. Cells and antibodies were incubated at 37° C. in 5% CO 2 incubator for 18-24 hrs. Samples were collected then stained with LIVE/DEAD fixable Aqua Dead Cell Stain Kit (Thermo Fisher, Cat #L34957) and analyzed on a Miltenyi MACSQuant 16 flow cytometer. The % dead target cell was gated using FITC+ Live/dead+. The EC₅₀ value was calculated using Graphpad Prism 9.3.0.

In vivo efficacy and mechanistic studies. 8-week-old female transgenic B-hCCR8 mice (Biocytogen, Cat #110096) were used. 0.5×10⁶ MC-38 (Shanghai Shunran Biotechnology Co., Ltd., Cat #M023) cells in 100 μl PBS were inoculated to the left upper side of each mouse by subcutaneous injection after local hair shaving. Tumor growth and mouse body weight were monitored twice a week. For each individual tumor, the longest longitudinal diameter as length and the widest transverse diameter as width were measured by using a Traceable Digital Caliper (VWR, Cat #62379-531). Tumor volume (TV) was then calculated by the formula TV=[length×(width)²]/2. Mice were grouped randomly when averaged tumor volume reached 87-89 mm 3 and 109-111 mm 3 in the monotherapy and combination therapy/mechanistic studies, respectively. Each group contained 8 mice. In the monotherapy study, human IgG1 negative control, GS-1811 and SD-171467-afuc were made in a stock solution of 1 mg/mL. In the combination therapy/mechanistic study, anti-mouse PD-1 and SD-171467-afuc were made in a stock solution of 0.03 and 0.1 mg/mL, respectively. In addition, a mixture solution contained both anti-mouse PD-1 (0.03 mg/mL) and SD-171467-afuc (0.1 mg/mL) was used for the combination therapy/mechanistic study. For each mouse, the volume of administered vehicle control (PBS) or antibody was calculated by the formula Volume (μ1)=Mouse body weight (g)×10 μl/g. Antibody drug were administered via the intraperitoneal (i.p.) route twice a week during the first two weeks and the 5^{th} dosing one week after the 4^{th} one with a total number of 5 doses. Tumor growth was monitored for 14 days after initial drug treatment.

At the end of the combination therapy/mechanistic study (22 days after treatment), the tumor and spleen samples were collected from human PBS and 1 mg/kg SD-171467-afuc treatment groups and minced into small pieces in digestion buffer [DMEM media (Corning, Cat #MT10013CM) supplemented with 0.36 mg/mL of Collagenase type IV (MP Biomedicals, Cat #IC19511090) and 10 μg/mL of DNase I (STEMCELL Technologies Inc. Cat #07900)]. Samples were then incubated at 37° C. degree for 30-45 minutes then filtered through cell strainer (Corning, Cat #352340). The strained cell suspensions were centrifuged, with the supernatant discarded afterwards. Cell pellets were resuspended with red blood cell lysis buffer (BioLegend, Cat #420301) to remove the red blood cells. The remaining cell suspensions were then counted and recorded total cell numbers using an automated cell counter. 100 ul of cell suspensions were used for FACs analysis. Cell samples were stained with live/dead marker (Thermo Fisher, Cat #L34957) for 20 minutes at room temperature and were treated with mouse Fc-blocker (BioLegend, Cat #101302) then stained with antibodies cocktail [anti-mouse CD45.1-APC/Cy7 (BioLegend, Cat #103116), anti-mouse CD3-BV-605 (BioLegend, Cat #100237), anti-mouse CD4-FITC (BioLegend, Cat #130308), anti-CD8-mouse-APC (BioLegend, Cat #162305), anti-human CCR8-PE (BioLegend, Cat #365704 antibodies) for 30 minutes on ice. Samples are then fixed with FoxP3 fix/perm buffer (BioLegend, Cat #421401) and permeated with FoxP3 perm buffer (BioLegend, Cat #421402). Samples were then stained with anti-Foxp3-BV421 (BioLegend, Cat #126419) antibody and analyzed with Miltenyi MACSQuant 16 flow cytometer. CCR8+ Treg cell population was gated (Single cell/Live cell/CD45.1+/CD4+/Foxp3+/CCR8+).

Pharmacokinetic study. 8-week-old female transgenic B-hCCR8 mice (Biocytogen, Cat #110096) were used. 0.5×10⁶ MC-38 (Shanghai Shunran Biotechnology Co., Ltd., Cat #M023) cells in 100 μl PBS were inoculated to the left upper side of each mouse by subcutaneous injection after local hair shaving. Tumor bearing mice were i.p. administered with SD-171467-afuc at 10 mg/kg 14 days after tumor cell inoculation. Goldenrod Animal Lancet (3 mm, Medipoint) was applied to collect blood samples from facial veins of treated mice at 0 hr (pre-treatment), 0.25 hr, 0.5 hr, 1 hr, 2 hr, 4 hr, 8 hr, 18 hr, 24 hr, 48 hr, 96 hr, 192 hr, 384 hr, and 576 hr (end point). Sixty μL of blood was collected by using BD Microtainer Tubes with heparin coating. After sitting on ice for 30-60 min, serum was separated by centrifugation at 5,000 rpm×5 min in 4° C., transferred into 2 mL cryogenic vials (Heathraw Scientific, Cat #HS23202A), snap frozen in ethanol with dry ice, and stored at −80° C. for downstream analysis. The concentration of drug in the serum was measured using the Human/NHP IgG quantification kit from Meso Scale Diagnostics (MSD, Cat #K150JLD) according to the kit instructions. Briefly, dilutions of animal serum were applied to a plate precoated with a human/NHP capture antibody and detected with an anti-IgG secondary.

Serum concentration of IgG was calculated by interpolation from a standard curve using the MSD Discovery Workbench software. These data were then used to perform a non-compartmental PK analysis using Certara Phoenix™ WinNonLin.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (0, or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. An anti-CCR8 antibody or antigen binding fragment thereof, wherein the antibody or antigen binding fragment comprises:
a heavy chain variable domain (VH) complementarity determining region (CDR) 1 comprising an amino acid sequence of any one of the following SEQ ID NOS: 1, 49, 67, 73, 79, or 85; and a VH CDR2 comprising the amino acid sequence of any one of the following SEQ ID NOS: 2, 50, 68, 74, 80, or 86; and a VH CDR3 comprising the amino acid sequence of any one of the following SEQ ID NOS: 3, 51, 69, 75, 81, or 87; and
a light chain variable domain (VL) CDR1 comprising an amino acid sequence of any one of the following SEQ ID NOS: 4, 52, 55, 58, 61, 64, 70, 76, 82, or 88; and a VL CDR2 comprising the amino acid sequence of any one of the following SEQ ID NOS: 5, 53, 56, 59, 62, 65, 71, 77, 83, or 89; and a VL CDR3 comprising the amino acid sequence of any one of the following SEQ ID NOS: 6, 54, 57, 60, 63, 66, 72, 78, 84, or 90.

2. The antibody or antigen binding fragment of claim 1, wherein the antibody comprises:
a VH comprising the amino acid sequence of any one of the following SEQ ID NOS: 7, 8, 9, 10, 11, 12, 13, 14, or 15, respectively; and
a VL comprising the amino acid sequence of any one of the following SEQ ID NOS: 16, 17, 18, 19, 20, 21, 22, 23, or 24, respectively.

3. The antibody or antigen binding fragment of claim 1, wherein the antibody comprises:
a VH encoded by a nucleic acid sequence having at least 95, 96, 97, 98, 99, or 100% sequences identity to any one of the following SEQ ID NOS: 25, 26, 27, 28, 29, 30, 31, 32, or 33; and
a VL encoded by a nucleic acid sequence having at least 95, 96, 97, 98, 99, or 100% sequences identity to any one of the following SEQ ID NOS: 34, 35, 36, 37, 18, 29, 40, 41, or 42.

4. The antibody or antigen binding fragment of claim 1, wherein the antibody is a monoclonal, bispecific, multivalent, multi-specific, diabody, chimeric, scFv antibody, or binding fragments thereof.

5. The antibody or antigen binding fragment of claim 4, wherein the antibody is fused to an Fc domain of any one of the following: human IgG1, human IgG2, human IgG3, and human IgG4.

6. The antibody or binding fragment of claim 1, wherein the antibody is a full-length antibody that is afucosylated.

7. The antibody or antigen binding fragment of claim 1, wherein the nucleic acid sequence is optimized for expression in a bacterial, fungal, mammalian, insect, or plant cell.

8. The antibody or antigen binding fragment of claim 1, wherein the antibody or antigen binding fragment does not bind CCR4.

9. The antibody or antigen binding fragment of claim 1, wherein the heavy chain CDRs are SEQ ID NOS: 1, 2 and 3, and the light chain CDRs are SEQ ID NOS: 4, 5, and 6.

10. A method of treating a disease in a subject in need thereof, comprising administering to the subject a therapeutically effective amount of the antibody of any one of claims 1 to 9.

11. The method of claim 10, where the disease is cancer.

12. The method of claim 11, where the cancer is infiltrated with regulatory T cells.

13. The method of claim 11, wherein the subject is human.

14. A method of making an anti-CCR8 antibody or antigen binding fragment comprising expressing in the antibody or antigen binding fragment in a cell or in vitro, wherein the antibody or antigen binding fragment comprises:
a heavy chain variable domain (VH) complementarity determining region (CDR) 1 comprising an amino acid sequence of any one of the following SEQ ID NOS: 1, 49, 67, 73, 79, or 85; and a VH CDR2 comprising the amino acid sequence of any one of the following SEQ ID NOS: 2, 50, 68, 74, 80, or 86; and a VH CDR3 comprising the amino acid sequence of any one of the following SEQ ID NOS: 3, 51, 69, 75, 81, or 87; and
a light chain variable domain (VL) CDR1 comprising an amino acid sequence of any one of the following SEQ ID NOS: 4, 52, 55, 58, 61, 64, 70, 76, 82, or 88; and a VL CDR2 comprising the amino acid sequence of any one of the following SEQ ID NOS: 5, 53, 56, 59, 62, 65, 71, 77, 83, or 89; and a VL CDR3 comprising the amino acid sequence of any one of the following SEQ ID NOS: 6, 54, 57, 60, 63, 66, 72, 78, 84, or 90.

15. The method of claim 14, where the cell is a bacterial, fungal, human, plant, or insect cell.

16. The method of claim 14, wherein the antibody is a monoclonal, bispecific, multivalent, multi-specific, diabody, chimeric, scFv antibody, or fragments thereof.

17. The method of claim 14, wherein the antibody or antigen binding fragment is afucosylated.

18. The method of claim 14, wherein the antibody or antigen binding fragment does not bind CCR4.

19. A nucleic acid comprising an anti-CCR8 antibody or antigen binding fragment comprising:
a heavy chain variable domain encoding polynucleotide having at least 95, 96, 97, 98, 99, or 100% sequence identify to SEQ ID NOS: 25, 26, 27, 28, 29, 30, 31, 32, or 33; and
a light chain variable domain encoding polynucleotide having at least 95, 96, 97, 98, 99, or 100% sequence identify to SEQ ID NOS: 34, 35, 36, 37, 38, 39, 40 41, or 42.

20. The nucleic acid of claim 19, wherein the antibody is a monoclonal, bispecific, multivalent, multi-specific, diabody, chimeric, scFv antibody, or fragments thereof.

21. The nucleic acid of claim 20, wherein an antibody binding domain is fused to an Fc domain of any one of the following: human IgG1, human IgG2, human IgG3, and human IgG4.

22. The nucleic acid of claim 19, wherein a nucleic acid sequence is optimized for expression in a bacterial, fungal, mammalian, insect, or plant cell.

23. The nucleic acid of claim 19, wherein the antibody or antigen binding fragment does not bind CCR4.

24. A vector comprising the nucleic acid of claim 19.

25. A host cell comprising nucleic acid the vector of claim 24.


SEQUENCE LISTING (94 sequences)

SEQ ID NO: 1 - protein, 8 aa, synthetic construct
  REGION 1..8: note = Synthetic: 1E6H2K - HC, CDR1
GFTFNTNA

SEQ ID NO: 2 - protein, 10 aa, synthetic construct
  REGION 1..10: note = Synthetic: 1E6H2K - HC, CDR2
IRSKSNNYAT

SEQ ID NO: 3 - protein, 15 aa, synthetic construct
  REGION 1..15: note = Synthetic: 1E6H2K - HC, CDR3
VRGKDTSGSYYAMDY

SEQ ID NO: 4 - protein, 11 aa, synthetic construct
  REGION 1..11: note = Synthetic: 1E6H2K - LC, CDR1
KSLLHSNGNTY

SEQ ID NO: 5 - skipped

SEQ ID NO: 6 - protein, 9 aa, synthetic construct
  REGION 1..9: note = Synthetic: 1E6H2K - LC, CDR3
MQHLEYPFT

SEQ ID NO: 7 - protein, 124 aa, synthetic construct
  REGION 1..124: note = Synthetic: SD-356253_VH
QVQLVETGGGLVQPKGSLKLSCAASGFTFNTNAMNWVRQAPGKGLEWLARIRSKSNNYATYYADSVKDRFTISRDDSQSMLYLQMKNLKSEDTAMYYCVRGKDTSGSYYAMDYWGQGTSVTVSS

SEQ ID NO: 8 - protein, 124 aa, synthetic construct
  REGION 1..124: note = Synthetic: SD-171467_VH
QVQLVESGGGLVQPGGSLKLSCAASGFTFNTNAMNWVRQASGKGLEWVGRIRSKSNNYATYYADSVKDRFTISRDDSKNTLYLQMNSLKTEDTAVYYCVRGKDTSGSYYAMDYWGQGTTVTVSS

SEQ ID NO: 9 - protein, 124 aa, synthetic construct
  REGION 1..124: note = Synthetic: SD-962141_VH
EVQLVESGGGLVQPGGSLKLSCAASGFTFNTNAMNWVRQAPGKGLEWVGRIRSKSNNYATYYADSVKDRFTISRDDSKSTLYLQMNSLKTEDTAVYYCVRGKDTSGSYYAMDYWGQGTLVTVSS

SEQ ID NO: 10 - protein, 124 aa, synthetic construct
  REGION 1..124: note = Synthetic: SD-611534_VH
EVQLVESGGGLVQPGGSLKLSCAASGFTFSTNAMNWVRQAPGKGLEWVGRIRSKSNNYATYYADSVKDRFTISRDDSKNTLYLQMNSLKTEDTAVYYCVRGKDTSGSYYAMDYWGQGTLVTVSS

SEQ ID NO: 11 - protein, 124 aa, synthetic construct; identical to SEQ ID NO: 8
  REGION 1..124: note = Synthetic: SD-580600_VH

SEQ ID NO: 12 - protein, 124 aa, synthetic construct
  REGION 1..124: note = Synthetic: SD-388950_VH
QVQLVESGGGLVQPGGSLKLSCAASGFTFNTNAMNWVRQASGKGLEWVGRIRSKSNNYATYYADSVKDRFTISRDDSQSMLYLQMNSLKTEDTAVYYCVRGKDTSGSYYAMDYWGQGTTVTVSS

SEQ ID NO: 13 - protein, 124 aa, synthetic construct; identical to SEQ ID NO: 8
  REGION 1..124: note = Synthetic: SD-117248_VH

SEQ ID NO: 14 - protein, 124 aa, synthetic construct
  REGION 1..124: note = Synthetic: SD-166154_VH
EVQLVESGGGLVQPGGSLKLSCAASGFTFSTNAMNWVRQASGKGLEWVGRIRSKSNNYATYYADSVKDRFTISRDDSKNTAYLQMNSLKTEDTAVYYCTRGKDTSGSYYAMDYWGQGTMVTVSS

SEQ ID NO: 15 - protein, 124 aa, synthetic construct; identical to SEQ ID NO: 14
  REGION 1..124: note = Synthetic: SD-969683_VH

SEQ ID NO: 16 - protein, 112 aa, synthetic construct
  REGION 1..112: note = Synthetic: SD-356253_VL
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIR

SEQ ID NO: 17 - protein, 112 aa, synthetic construct
  REGION 1..112: note = Synthetic: SD-171467_VL
DIVMTQSPLSLPVTPGEPASISCRSSKSLLHSNGNTYLYWFLQKPGQSPQLLIYRMSNLASGVPDRFSGSGSGTDFTLKISRVEAEDVGVYYCMQHLEYPFTFGGGTKVEIK

SEQ ID NO: 18 - protein, 112 aa, synthetic construct; identical to SEQ ID NO: 17
  REGION 1..112: note = Synthetic: SD-962141_VL

SEQ ID NO: 19 - protein, 112 aa, synthetic construct; identical to SEQ ID NO: 17
  REGION 1..112: note = Synthetic: SD-611534_VL

SEQ ID NO: 20 - protein, 112 aa, synthetic construct
  REGION 1..112: note = Synthetic: SD-580600_VL
DIVMTQSALSLPVTPGEPASISCRSSKSLLHSNGNTYLYWFLQKPGQSPQLLIYRMSNLASGVPDRFSGSGSGTDFTLKISRVEAEDVGVYYCMQHLEYPFTFGGGTKVEIK

SEQ ID NO: 21 - protein, 112 aa, synthetic construct; identical to SEQ ID NO: 20
  REGION 1..112: note = Synthetic: SD-388950_VL

SEQ ID NO: 22 - protein, 112 aa, synthetic construct
  REGION 1..112: note = Synthetic: SD-117248_VL
DIVMTQSPLSLPVTPGEPASISCRSSKSLLHSNGNTYLYWFLQKPGQSPQLLIYRMSNLASGVPDRFSGSGSGTAFTLKISRVEAEDVGVYYCMQHLEYPFTFGGGTKVEIK

SEQ ID NO: 23 - protein, 112 aa, synthetic construct; identical to SEQ ID NO: 22
  REGION 1..112: note = Synthetic: SD-166154_VL

SEQ ID NO: 24 - protein, 112 aa, synthetic construct
  REGION 1..112: note = Synthetic: SD-969683_VL
DIVMTQSPLSLPVTPGESVSISCRSSKSLLHSNGNTYLYWFLQKPGQSPQLLIYRMSNLASGVPDRFSGSGSGTDFTLRISRVEAEDVGVYYCMQHLEYPFTFGGGTKVEIK

SEQ ID NO: 25 - DNA, 372 bp, synthetic construct
  misc_feature 1..372: note = Synthetic: SD-356253_VH
  source 1..372: mol_type = other DNA
caagtgcagctcgtggaaactggcggcggactggtgcagccgaagggtagcctgaagctgtcctgtgcggcctccggtttcacttttaacaccaacgccatgaattgggtccgccaggcacccgggaaagggctggaatggctcgcgagaatccggtccaagtcgaacaactacgccacctactacgctgactcggtcaaggacaggttcacgatttcccgcgacgattcacagagcatgctgtaccttcaaatgaagaacttgaagtcagaggataccgccatgtactattgcgtgcggggaaaggacacctccggatcctactacgccatggactactggggacagggcaccagcgtgactgtgtcctcg

SEQ ID NO: 26 - DNA, 372 bp, synthetic construct
  misc_feature 1..372: note = Synthetic: SD-171467_VH
  source 1..372: mol_type = other DNA
caggtccagctggtagagtccggcggcggactggtccagcctggcggaagtctgaaattgtcttgcgctgctagcgggttcactttcaatacaaatgcaatgaactgggtgcgccaggcaagtggcaaaggactggagtgggtcggaagaatcagatccaagagcaataactacgccacttactatgccgactccgttaaggatcgcttcacgatatcccgcgacgacagtaagaatactctctacctgcagatgaactccttaaaaacagaagacactgccgtatactactgtgtaagaggtaaagacacctcaggctcctattatgccatggactattggggacagggtacaaccgtcacagtgtccagc

SEQ ID NO: 27 - DNA, 372 bp, synthetic construct
  misc_feature 1..372: note = Synthetic: SD-962141_VH
  source 1..372: mol_type = other DNA
gaagtgcagctggttgaatccggcggggggctggtgcagcctggaggctccctcaagctctcttgcgctgcctccggatttaccttcaatacaaatgcgatgaactgggtgcgccaggcacctgggaaaggtttggagtgggtaggccgcatacgcagcaagagcaataactatgccacctattatgcagatagcgtaaaagacagattcactatcagtcgggacgactctaaatccaccctctatctgcagatgaactcactgaagactgaggacaccgctgtctattactgcgtgcggggcaaggatacgtctggtagctattatgctatggactattggggccaggggacactggtcacggtttcttct

SEQ ID NO: 28 - DNA, 372 bp, synthetic construct
  misc_feature 1..372: note = Synthetic: SD-611534_VH
  source 1..372: mol_type = other DNA
gaggtgcagctggtagagtctgggggagggttagtccagccggggggttccctgaagctcagctgtgccgcctccgggtttacattttccaccaacgctatgaactgggtaagacaggctcccggaaagggactcgaatgggtgggccgcattcgatcaaagagcaacaactatgccacgtactacgcggactcagtaaaagatcgcttcactatatccagagatgactctaaaaacaccttgtacctgcagatgaactcacttaaaactgaagacactgctgtctactactgcgtaaggggcaaggacacatccggcagctattatgccatggattactggggtcaaggtaccctcgttacagtgagcagc

SEQ ID NO: 29 - DNA, 372 bp, synthetic construct; identical to SEQ ID NO: 26
  misc_feature 1..372: note = Synthetic: SD-580600_VH
  source 1..372: mol_type = other DNA

SEQ ID NO: 30 - DNA, 372 bp, synthetic construct
  misc_feature 1..372: note = Synthetic: SD-388950_VH
  source 1..372: mol_type = other DNA
caagtgcaattggtcgagtccggcgggggcctggtgcaacctggcgggagcctgaagctctcctgcgcggcaagcgggtttacttttaacaccaatgccatgaattgggttaggcaagcgtccggtaagggattggagtgggtggggaggatccgatcaaaatctaataattacgccacctattacgcagattctgtcaaagatcgctttactatctcaagagatgacagtcagagtatgctctatctgcaaatgaactccctgaagacggaagacacagctgtttactattgtgtgcgggggaaggacactagtggtagctattacgccatggactactggggtcagggaaccacagtcaccgtgagctct

SEQ ID NO: 31 - DNA, 372 bp, synthetic construct; identical to SEQ ID NO: 26
  misc_feature 1..372: note = Synthetic: SD-117248_VH
  source 1..372: mol_type = other DNA

SEQ ID NO: 32 - DNA, 372 bp, synthetic construct
  misc_feature 1..372: note = Synthetic: SD-166154_VH
  source 1..372: mol_type = other DNA
gaggtgcagttggtcgagagtggtgggggacttgtacagcctgggggctcactgaaattatcctgtgcggcgagtggatttactttttctactaatgctatgaattgggttcgccaggcgagcggaaaaggcctggaatgggtcgggaggattcgctctaaatctaacaactatgcaacctactatgccgactccgttaaggatcgctttactataagcagagatgacagcaaaaacacagcgtatctgcaaatgaacagcctcaaaacagaggacacagccgtttactactgtactagaggcaaagacactagcggtagttactatgccatggattactggggtcaggggacgatggttaccgtgagttcc

SEQ ID NO: 33 - DNA, 372 bp, synthetic construct; identical to SEQ ID NO: 32
  misc_feature 1..372: note = Synthetic: SD-969683_VH
  source 1..372: mol_type = other DNA

SEQ ID NO: 34 - DNA, 336 bp, synthetic construct
  misc_feature 1..336: note = Synthetic: SD-356253_VL
  source 1..336: mol_type = other DNA
gacatcgtgatgactcaggccgccccttccgtgcccgtcaccccgggagaatccgtgtcaatctcgtgtcggtcctccaagtcgctgcttcattccaacgggaacacctatctgtactggttcttgcaaagacccggacagagcccgcagctgctgatctaccgcatgtctaacctcgctagcggagtgccagatcggttttcgggatccggcagcggcaccgcgttcactctgcgcatttcaagggtcgaagccgaggacgtgggcgtgtactactgcatgcagcacctggagtaccctttcaccttcgggtccggtaccaagctcgaaattcgg

SEQ ID NO: 35 - DNA, 336 bp, synthetic construct
  misc_feature 1..336: note = Synthetic: SD-171467_VL
  source 1..336: mol_type = other DNA
gatattgtgatgactcagtcgccgctgtcattgcccgtgacccctggagaacccgcgtcgatttcctgtcggtcctccaagtccctcctgcactccaacgggaacacctacctctactggtttctgcaaaagccgggacagagcccacagctgctgatctatcggatgagcaaccttgcctcgggagtgcctgaccgcttctccggaagcggctcagggaccgacttcaccctgaaaatctccagagtggaggccgaagatgtcggcgtgtactactgcatgcagcatctggagtacccgttcactttcggcggtggtaccaaggtcgaaatcaag

SEQ ID NO: 36 - DNA, 336 bp, synthetic construct; identical to SEQ ID NO: 35
  misc_feature 1..336: note = Synthetic: SD-962141_VL
  source 1..336: mol_type = other DNA

SEQ ID NO: 37 - DNA, 336 bp, synthetic construct; identical to SEQ ID NO: 35
  misc_feature 1..336: note = Synthetic: SD-611534_VL
  source 1..336: mol_type = other DNA

SEQ ID NO: 38 - DNA, 336 bp, synthetic construct
  misc_feature 1..336: note = Synthetic: SD-580600_VL
  source 1..336: mol_type = other DNA
gacattgttatgacccagtctgccttatctctgcccgtaacccctggggagcccgcgagcataagttgcaggtcctcaaaaagccttttgcatagtaatggcaatacatacttgtattggtttctgcagaagcctggacagagccctcagctgctgatttatagaatgagcaacctcgcttctggagtgcccgaccgctttagcggctctggatcagggactgattttacactgaagataagcagagtggaagctgaggatgttggcgtgtattactgtatgcagcatctcgagtatccatttacatttggtgggggcactaaggtggaaatcaag

SEQ ID NO: 39 - DNA, 336 bp, synthetic construct; identical to SEQ ID NO: 38
  misc_feature 1..336: note = Synthetic: SD-388950_VL
  source 1..336: mol_type = other DNA

SEQ ID NO: 40 - DNA, 336 bp, synthetic construct
  misc_feature 1..336: note = Synthetic: SD-117248_VL
  source 1..336: mol_type = other DNA
gatatcgtcatgactcagagccccctgtccctgccggtgacccccggcgagccagcgtccatttcctgtcgcagctctaaatcactgctgcattctaatggcaatacctacttgtactggttccttcagaaaccaggccagtcaccacagctgctgatatatcgtatgtctaatttggcgagcggcgtgcctgaccgattctcaggctctggcagcggcactgcttttaccctgaagattagtcgggtcgaggccgaagatgtgggagtctactactgtatgcagcatctggaatacccttttacttttgggggcggcactaaggtagagattaaa

SEQ ID NO: 41 - DNA, 336 bp, synthetic construct; identical to SEQ ID NO: 40
  misc_feature 1..336: note = Synthetic: SD-166154_VL
  source 1..336: mol_type = other DNA

SEQ ID NO: 42 - DNA, 336 bp, synthetic construct
  misc_feature 1..336: note = Synthetic: SD-969683_VL
  source 1..336: mol_type = other DNA
gacatagtgatgacccagagtccattatccctgccagtcacgcctggggagtcagtgagcatttcctgccgatccagtaaaagcctccttcattccaatggtaatacatacctgtactggttcctccagaaacccggccaatctcctcagttacttatttacagaatgtccaacctcgcatctggggtgcccgacaggttttccggaagtgggtcaggcactgacttcaccctgaggatcagtagggtggaggccgaggatgtgggcgtgtactactgcatgcagcatctcgagtaccctttcaccttcgggggaggaacaaaagtagagataaaa

SEQ ID NO: 43 - protein, 8 aa, synthetic construct
  REGION 1..8: note = Synthetic: 1E6H3K - HC, CDR1
GFTFNTNA

SEQ ID NO: 44 - protein, 10 aa, synthetic construct
  REGION 1..10: note = Synthetic: 1E6H3K - HC, CDR2
IRSKSNNYAT

SEQ ID NO: 45 - protein, 15 aa, synthetic construct
  REGION 1..15: note = Synthetic: 1E6H3K - HC, CDR3
VRGKDTSGSYYAMDY

SEQ ID NO: 46 - protein, 11 aa, synthetic construct
  REGION 1..11: note = Synthetic: 1E6H3K - LC, CDR1
KSLLHSNGNTY

SEQ ID NO: 47 - skipped

SEQ ID NO: 48 - protein, 9 aa, synthetic construct
  REGION 1..9: note = Synthetic: 1E6H3K - LC, CDR3
MQHLEYPFT

SEQ ID NO: 49 - protein, 8 aa, synthetic construct
  REGION 1..8: note = Synthetic: 7B_600_030 -HC, CDR1
GFIFETYA

SEQ ID NO: 50 - protein, 10 aa, synthetic construct
  REGION 1..10: note = Synthetic: 7B_600_030 -HC, CDR2
IRSKSNNYAT

SEQ ID NO: 51 - protein, 12 aa, synthetic construct
  REGION 1..12: note = Synthetic: 7B_600_030 -HC, CDR3
VRGLLRYRFFDV

SEQ ID NO: 52 - protein, 11 aa, synthetic construct
  REGION 1..11: note = Synthetic: 7B_600_030 - LC, CDR1
KSLLHSNGNTY

SEQ ID NO: 53 - skipped

SEQ ID NO: 54 - protein, 9 aa, synthetic construct
  REGION 1..9: note = Synthetic: 7B_600_030 - LC, CDR3
MQHLEYPFT

SEQ ID NO: 55 - protein, 8 aa, synthetic construct
  REGION 1..8: note = Synthetic: 7B_000_032, CDR1
GFTFNTYA

SEQ ID NO: 56 - protein, 10 aa, synthetic construct
  REGION 1..10: note = Synthetic: 7B_000_032, CDR2
IRSKSNNYAT

SEQ ID NO: 57 - protein, 12 aa, synthetic construct
  REGION 1..12: note = Synthetic: 7B_000_032, CDR3
VRGLLRYRFFDV

SEQ ID NO: 58 - protein, 11 aa, synthetic construct
  REGION 1..11: note = Synthetic: 7B_000_032, CDR1
KSLLHSNGNTY

SEQ ID NO: 59 - skipped

SEQ ID NO: 60 - protein, 9 aa, synthetic construct
  REGION 1..9: note = Synthetic: 7B_000_032, CDR3
QQHTNWPFT

SEQ ID NO: 61 - protein, 8 aa, synthetic construct
  REGION 1..8: note = Synthetic: 7B_600_022, CDR1
GFIFETYA

SEQ ID NO: 62 - protein, 10 aa, synthetic construct
  REGION 1..10: note = Synthetic: 7B_600_022, CDR2
IRSKSNNYAT

SEQ ID NO: 63 - protein, 12 aa, synthetic construct
  REGION 1..12: note = Synthetic: 7B_600_022, CDR3
VRGLLRYRFFDV

SEQ ID NO: 64 - protein, 11 aa, synthetic construct
  REGION 1..11: note = Synthetic: 7B_600_022, CDR1
KSLLHSNGNTY

SEQ ID NO: 65 - skipped

SEQ ID NO: 66 - protein, 9 aa, synthetic construct
  REGION 1..9: note = Synthetic: 7B_600_022, CDR3
QQHTNWPFT

SEQ ID NO: 67 - protein, 8 aa, synthetic construct
  REGION 1..8: note = Synthetic: 7B_600_032 - HC, CDR1
GFIFETYA

SEQ ID NO: 68 - protein, 10 aa, synthetic construct
  REGION 1..10: note = Synthetic: 7B_600_032 - HC, CDR2
IRSKSNNYAT

SEQ ID NO: 69 - protein, 12 aa, synthetic construct
  REGION 1..12: note = Synthetic: 7B_600_032 - HC, CDR3
VRGLLRYRFFDV

SEQ ID NO: 70 - protein, 11 aa, synthetic construct
  REGION 1..11: note = Synthetic: 7B_600_032 - LC, CDR1
KSLLHSNGNTY

SEQ ID NO: 71 - skipped

SEQ ID NO: 72 - protein, 9 aa, synthetic construct
  REGION 1..9: note = Synthetic: 7B_600_032 - LC, CDR3
QQHTNWPFT

SEQ ID NO: 73 - protein, 8 aa, synthetic construct
  REGION 1..8: note = Synthetic: 7B_600_012 - HC, CDR1
GFIFETYA

SEQ ID NO: 74 - protein, 10 aa, synthetic construct
  REGION 1..10: note = Synthetic: 7B_600_012 - HC, CDR2
IRSKSNNYAT

SEQ ID NO: 75 - protein, 12 aa, synthetic construct
  REGION 1..12: note = Synthetic: 7B_600_012 - HC, CDR3
VRGLLRYRFFDV

SEQ ID NO: 76 - protein, 11 aa, synthetic construct
  REGION 1..11: note = Synthetic: 7B_600_012 - LC, CDR1
KSLLHSNGNTY

SEQ ID NO: 77 - skipped

SEQ ID NO: 78 - protein, 9 aa, synthetic construct
  REGION 1..9: note = Synthetic: 7B_600_012 - LC, CDR3
QQHTNWPFT

SEQ ID NO: 79 - protein, 8 aa, synthetic construct
  REGION 1..8: note = Synthetic: 7B_600_020 - HC, CDR1
GFIFETYA

SEQ ID NO: 80 - protein, 10 aa, synthetic construct
  REGION 1..10: note = Synthetic: 7B_600_020 - HC, CDR2
IRSKSNNYAT

SEQ ID NO: 81 - protein, 12 aa, synthetic construct
  REGION 1..12: note = Synthetic: 7B_600_020 - HC, CDR3
VRGLLRYRFFDV

SEQ ID NO: 82 - protein, 11 aa, synthetic construct
  REGION 1..11: note = Synthetic: 7B_600_020 - LC, CDR1
KSLLHSNGNTY

SEQ ID NO: 83 - skipped

SEQ ID NO: 84 - protein, 9 aa, synthetic construct
  REGION 1..9: note = Synthetic: 7B_600_020 - LC, CDR3
MQHLEYPFT

SEQ ID NO: 85 - protein, 8 aa, synthetic construct
  REGION 1..8: note = Synthetic: K17_050_050 - HC, CDR1
GYTFTSYN

SEQ ID NO: 86 - protein, 8 aa, synthetic construct
  REGION 1..8: note = Synthetic: K17_050_050 - HC, CDR2
VHPGSGST

SEQ ID NO: 87 - protein, 10 aa, synthetic construct
  REGION 1..10: note = Synthetic: K17_050_050 - HC, CDR3
ARKGGTPFAY

SEQ ID NO: 88 - protein, 11 aa, synthetic construct
  REGION 1..11: note = Synthetic: K17_050_050 -LC, CDR1
QSLVHSNAATY

SEQ ID NO: 89 - skipped

SEQ ID NO: 90 - protein, 9 aa, synthetic construct
  REGION 1..9: note = Synthetic: K17_050_050 -LC, CDR3
SQSTHVPYT

SEQ ID NO: 91 - protein, 7 aa, synthetic construct
  REGION 1..7: note = Synthetic: extracellular loop 1
DQWVFGT

SEQ ID NO: 92 - protein, 10 aa, synthetic construct
  REGION 1..10: note = Synthetic: extracellular loop 2
VASEDGVLQC

SEQ ID NO: 93 - protein, 54 aa, synthetic construct
  REGION 1..54: note = Synthetic: CCR8 MEM
SPEIKKLIEQIKSDQWVFGTEACNEIQTLLKEESGPAKIEVASEDGVLQCRIVF

SEQ ID NO: 94 - protein, 5 aa, synthetic construct
  REGION 1..5: note = Synthetic: linker sequence
GGGGS